(19) United States
(12) Patent Application Publication
(10) Pub. No.: US 2024/0272176 A1
(43) Pub. Date: Aug. 15, 2024

(54) SINGLE DOMAIN ANTIBODY SPECIFIC FOR PHOSPHORYLATED H2AX AND ITS USES

(71) Applicants: UNIVERSITE DE STRASBOURG, STRASBOURG (FR); INSTITUT PASTEUR, PARIS CEDEX 15 (FR); CENTRE NATIONAL DE LA RECHERCHE SCIENTIFIQUE, PARIS (FR)

(72) Inventors: ETIENNE WEISS, FEGERSHEIM (FR); ERIC MOEGLIN, LIÈGE (BE); PIERRE LAFAYE, PARIS (FR)

(21) Appl. No.: 18/290,158

(22) PCT Filed: May 11, 2022
(86) PCT No.: PCT/EP2022/062831

§ 371 Date: Nov. 10, 2023

(30) Foreign Application Priority Data
May 12, 2021 (EP) 21305627.8

(51) Int. Cl. G01N 33/68; C07K 16/18

(52) U.S. Cl. CPC G01N 33/6875; C07K 16/18; C07K 2317/33; C07K 2317/35; C07K 2317/569; C07K 2319/60; G01N 2440/14

(57) ABSTRACT

The present invention relates to a single domain antibody directed against H2AX with a phosphorylation of serine at position 139 (γ-H2AX), a bivalent molecule comprising said single domain antibody and their use for detecting DNA damage and/or DNA replication stress.

### FIELD OF THE INVENTION

The present invention relates to an antibody specific for γ-H2AX and its uses as a laboratory tool.

### BACKGROUND OF THE INVENTION

Histones constitute the core proteins of chromatin and their post-translational modifications (PTMs) contribute to the molecular basis of epigenetic gene regulation and cellular memory. In humans, several variant forms of histones have been described and this is particularly relevant for the H2A histone. The H2A variants represent the largest and most diverse family of histones; there is overwhelming evidence that their unstructured N- and C-termini, which protrude out of the core structure of the nucleosome, harbor several sites for PTMs in response to varying stimuli. The H2AX variant shares high amino acid similarity with H2A and is characterized by an extended C-terminus, which is phosphorylated when the cells become injured by agents that provoke DNA replication stress (RS) and genome instability. The phosphorylation of serine at position 139 (S139) of H2AX has been particularly well studied and represents a key event in the detection and response to DNA damage.

Phosphorylation of histone H2AX at S139, which gives rise to what is generally referred to as γ-H2AX, is in fact a very early step in the DNA damage response (DDR) and an essential signal for the recruitment and retention of DDR complexes at the site of damage. Three different phosphatidylinositol 3 kinase (PI3K)-related kinases mediate S139 phosphorylation on H2AX: ATM (ataxia-telangiectasia mutated), ATR (ATM and Rad3-related), and DNA-PK (DNA-dependent protein kinase). ATM and DNA-PK share functional redundancy upon ionizing radiation, while ATR may preferentially phosphorylate H2AX during RS. This PTM of H2AX is highly dynamic and a number of phosphatases, including those of the PPP family and Wip1, are able to dephosphorylate γ-H2AX to fine-tune the duration and intensity of the DDR signaling. It has also been found that H2AX can be phosphorylated at the threonine residue at position 136 (T136) and at the C-terminal tyrosine residue at position 142 (Y142) to facilitate DNA repair, whereas the persistency of the latter PTM may also trigger apoptosis. Nevertheless, S139 phosphorylation is regarded as the main PTM of H2AX since it is specifically recognized by the adaptor protein MDC1, which further recruits several E3 ubiquitin ligases to favor DNA repair and/or restart of the halted forks during RS.

Because γ-H2AX is involved in the DDR, it is generally considered a biomarker of DNA double-strand breaks (DSBs) and its relevance as read-out of sustained RS is well accepted. In addition, H2AX is also phosphorylated in the absence of DNA breakage, likely during replication fork arrest and subsequent single-stranded DNA accumulation, and this early event upon insult induces the formation of discrete nuclear foci of γ-H2AX, which can be visualized with specific antibodies under the microscope. The formation of γ-H2AX, which can spread progressively over the whole nucleus (pan-nuclear γ-H2AX) following chromatin modification by loop extrusion, gives in fact an estimate of the severity of the RS. γ-H2AX is considered nowadays a universal bio-indicator of the severity of genotoxic compounds that interfere with DNA replication in vitro and in vivo. γ-H2AX is also an early biomarker in clinics to check for tissue health status after radiotherapy, chemotherapy or radiation treatment. Indeed, almost all studies aiming at selecting small molecules triggering irreversible genome instability refer to γ-H2AX formation and retention to assess their potency. In particular, tracking γ-H2AX is of high interest for validating chemotherapeutics and for controlling the carcinogenic properties of chemicals present in biological samples. From a drug discovery point of view, this biomarker is of great interest to screen for efficacy and toxicity of therapeutic treatments. Immunofluorescence with validated antibodies remains so far the method of choice for accurately determining γ-H2AX levels; however, currently, there is no simple tool available for monitoring γ-H2AX turn-over and for measuring its direct impact on cell viability.

In a previous study, the inventors have shown that delivery in cells of antigen-binding fragments (Fabs) derived from an anti-γ-H2AX monoclonal antibody (mAb) allows following the fate of cancer cells after treatment with varying RS-inducing drug combinations (Moeglin et al, 2019, Cancers. 11:355. doi:10.3390/cancers11030355; Conic et al, 2018, J. Cell Biol. 217:1537-1552. doi:10.1083/jcb.201709153). Although it was possible to show with this method that extensive γ-H2AX phosphorylation is indicative of commitment to irreversible cell death, the inventors could not clearly identify the dynamic changes in the levels of γ-H2AX during the treatment. Indeed, conventional antibodies cannot be easily delivered in living cells, and therefore require fixation of the samples.

Recently, it has been shown that single-domain antibody fragments of camelids, generally termed nanobodies, represent exquisite tools for tracking intracellular molecules. Nanobodies correspond to the variable domain (VHH) of the heavy chain-only antibodies (HcAb) expressed in these animals. They can be cloned as VHH repertoires with minimal modification from total RNA of peripheral blood mononuclear cells (PBMCs) obtained after immunization, thus presenting an authentic picture of the in vivo-maturated heavy chain repertoire diversity. Moreover, their small size (˜15 kDa) compared to conventional antibodies (˜150 kDa) and, especially, their capacity to fold stably in a reducing environment make them excellent binding molecules in cells. While alpaca-derived nanobodies against γ-H2AX have already been generated (Rajan et al, 2015, FEBS Open Bio. 5:779-788. doi:10.1016/j.fob.2015.09.005), these tools didn't allow for the specific unambiguous detection of γ-H2AX in irradiated cancer cells.

In conclusion, Histone H2AX phosphorylated at serine 139 (γ-H2AX) is a hallmark of DNA damage, signaling the presence of DNA double-strand breaks and global replication stress in mammalian cells. While γ-H2AX can be visualized with antibodies in fixed cells, its detection in living cells was so far not possible. Therefore, there is still a need for tools specific for γ-H2AX suitable for in vivo use in living cells for detecting DNA Damage and replication stress.

More specifically, since upon insult, γ-H2AX levels vary from one cell to another, a reagent that would consent monitoring in individual cells both γ-H2AX levels and their fate after treatment with varying doses of genotoxic agents would be useful. Indeed, classical antibodies (i.e., IgG) have the disadvantages to be relatively expensive reagents and only the monovalent Fab format of IgG, that can be obtained following digestion with papain, diffuses freely into the nucleus upon delivery. In addition, detection of γ-H2AX with complete antibodies can only be carried out in fixed cells (end-point experiments) and thus does not allow to study transient dynamic states of the chromatin following damage. When compared to classical antibodies, single-domain antibodies can be easily produced in bacteria and the methodologies used for the selection gives access to their DNA sequence.

DETAILED DESCRITION OF THE INVENTION To develop an immunological probe able to detect and track γ-H2AX in living cells, the inventors had to perform numerous selection attempts to isolate one clone that could specifically interact with the peptide used for immunization, confirming that single-domain antibodies cannot easily bind to small linear epitopes. This might also be the reason why another group was unsuccessful in selecting an anti-γ-H2AX nanobody following immunization of a lama with the same peptide (Jullien et al, J. Cell Sci. 2016, 129, 2673-2683, doi:10.1242/jcs.183103). Finally, they have isolated single domain antibodies (called nanobodies) that are easily expressed as functional recombinant proteins and report the extensive characterization of a novel nanobody that specifically recognizes γ-H2AX. The interaction of this nanobody with the C-terminal end of γ-H2AX was solved by X-ray crystallography. Moreover, the inventors engineered a bivalent version of this nanobody and showed that bivalency is essential to quantitatively visualize γ-H2AX in fixed drug-treated cells. After labelling with a chemical fluorophore, the inventors were able to detect γ-H2AX in a single-step assay with the same sensitivity as with validated antibodies that are used with an assay having several steps. Then, the use of the nanobodies identified by the inventors allows an improved assay which is more cost-effective. Moreover, the inventors produced fluorescent nanobody fusion proteins and applied a transduction strategy to visualize with precision γ-H2AX foci present in intact living cells following drug treatment. Together, this novel tool allows performing fast screenings of genotoxic drugs and enables to study the dynamics of this particular chromatin modification in individual cells under a variety of conditions.

Accordingly, the present invention relates to a single domain antibody directed against H2AX with a phosphorylation of serine at position 139 (γ-H2AX) comprising a variable domain comprising three CDRs (complementarity determining regions), namely CDR1, CDR2 and CDR3, consisting in the amino acid sequence of SEQ ID NO: 1: GLT(L/F)SRYA for CDR1, the amino acid sequence of SEQ ID NO: 2: ITASGRTT for CDR2, and the amino acid sequence of SEQ ID NO: 3: AADYGX₁X₂X₃YTRRQSEYX₄Y for CDR3, wherein X₁ and X₂ are any amino acid, X₃ is K or R, and X₄ is D or E.

Optionally, CDR1 is GLTLSRYA. Preferably, CDR1 is GLTFSRYA.

Optionally, X₁ and X₂ are independently selected in the group consisting of A, V, S, N, K, R, T and G, especially of S, N, K, R, T and G. Preferably, X₁ is selected in the group consisting of S, N, T and G. Preferably, X₂ is selected in the group consisting of G, K and S.

Optionally, X₁ is selected in the group consisting of S, N, T and G; X₂ is selected in the group consisting of G, K and S; X₃ is K or R; and X₄ is D or E. Preferably, X₃ is R. Preferably, X₄ is E. Especially, X₃ is R and X₄ is E. Alternatively, X₃ is K and X₄ is D.

Optionally, the amino acid sequence of CDR3 can be selected in the following group:

(SEQ ID NO: 4)
AADYGSGKYTRRQSEYDY;
(SEQ ID NO: 5)
AADYGNKRYTRRQSEYEY;
(SEQ ID NO: 6)
AADYGTSRYTRRQSEYEY;
(SEQ ID NO: 7)
AADYGGGRYTRRQSEYEY;
(SEQ ID NO: 8)
AADYGSGRYTRRQSEYDY;
(SEQ ID NO: 9)
AADYGSGKYTRRQSEYEY;
(SEQ ID NO: 10)
AADYGSGRYTRRQSEYEY;
(SEQ ID NO: 11)
AADYGNKKYTRRQSEYEY;
(SEQ ID NO: 12)
AADYGNKRYTRRQSEYDY;
(SEQ ID NO: 13)
AADYGNKKYTRRQSEYDY;
(SEQ ID NO: 14)
AADYGTSKYTRRQSEYEY;
(SEQ ID NO: 15)
AADYGTSRYTRRQSEYDY;
(SEQ ID NO: 16)
AADYGTSKYTRRQSEYDY;
(SEQ ID NO: 17)
AADYGGGKYTRRQSEYEY;
(SEQ ID NO: 18)
AADYGGGRYTRRQSEYDY;
and
(SEQ ID NO: 19)
AADYGGGRYTRRQSEYEY.

In a particular aspect, the amino acid sequence of CDR3 is selected from the group consisting of

(SEQ ID NO: 5)
AADYGNKRYTRRQSEYEY;
(SEQ ID NO: 6)
AADYGTSRYTRRQSEYEY;
(SEQ ID NO: 11)
AADYGNKKYTRRQSEYEY;
(SEQ ID NO: 12)
AADYGNKRYTRRQSEYDY;
(SEQ ID NO: 13)
AADYGNKKYTRRQSEYDY;
(SEQ ID NO: 14)
AADYGTSKYTRRQSEYEY;
(SEQ ID NO: 15)
AADYGTSRYTRRQSEYDY;
and
(SEQ ID NO: 16)
AADYGTSKYTRROSEYDY.

In a more particular aspect, the amino acid sequence of CDR3 is selected from the group consisting of

(SEQ ID NO: 4)
AADYGSGKYTRRQSEYDY;
(SEQ ID NO: 5)
AADYGNKRYTRRQSEYEY;
(SEQ ID NO: 6)
AADYGTSRYTRRQSEYEY;
and
(SEQ ID NO: 7)
AADYGGGRYTRRQSEYEY.

and more particularly of

(SEQ ID NO: 5)
AADYGNKRYTRRQSEYEY
and;
(SEQ ID NO: 6)
AADYGTSRYTRRQSEYEY

In a very particular aspect, the amino acid sequence of CDR3 is AADYGTSRYTRRQSEYEY (SEQ ID NO: 6).

In a particular aspect, the single domain antibody is a VHH, preferably from Camelidae, more preferably from Llama species, or camelized framework regions of a human VH.

In a particular aspect, the single domain antibody is an antibody that comprises, consists in, or consists essentially in, the amino acid sequence of SEQ ID NO: 20 or a variant amino acid sequence having no more than 1, 2, 3, 4, 5, 6, 7, 8, 9, or 10 amino acid additions, deletions, substitutions, or combinations thereof within the sequence of SEQ ID NO: 20, said addition, deletion, or substitution being outside of CDR1, CDR2 and CDR3 (underlined in the sequence for convenience), wherein the amino acid sequence of SEQ ID NO: 20 is

(SEQ ID NO: 20)
MA(E/D)VQLXXSGGGXVQXG(G/D)SLRLSC(S/A)(A/T)SGLT(F/
L)SRYAMGWFRQAPGNEREFVAVITASGRTTLYADS(V/L)KGRFTISR
DNAKNTVALQMQSLKPEDTAVYYCAADYGX₁X₂X₃YTRRQSEYX₄YWGQG
TQVTVSS(X)ₙAAA,

with n being 0-10, preferably being 0 or 9, more preferably being 0; X being any amino acid; X₁ and X₂ being any amino acid, X₃ being K or R, and X₄ being D or E. In a particular aspect, X₁, X₂, X₃ and X₄ can be as defined in any particular aspect as described above.

In a particular aspect, the single domain antibody is an antibody that comprises, consists in, or consists essentially in, the amino acid sequence of SEQ ID NO: 21 or a variant amino acid sequence having no more than 1, 2, 3, 4, 5, 6, 7, 8, 9, or 10 amino acid additions, deletions, substitutions, or combinations thereof within the sequence of SEQ ID NO: 21, said addition, deletion, or substitution being outside of CDR1, CDR2 and CDR3 (underlined in the sequence for convenience), wherein the amino acid sequence of SEQ ID NO: 21 is

(SEQ ID NO: 21)
MA(E/D)VQLVESGGGLVQAGDSLRLSCA(A/T)SGLTFSRYAMGWFRQ
APGNEREFVAVITASGRTTLYADSVKGRFTISRDNAKNTVALQMQSLKP
EDTAVYYCAADYGX₁X₂RYTRRQSEYX₄YWGQGTQVTVSSAAA,

with X₁ and X₂ are any amino acid and X₄ being D or E. In a particular aspect, X₁ and X₂ can be as defined in any particular aspect as described above.

In a particular aspect, the single domain antibody is an antibody that comprises, consists in, or consists essentially in, the amino acid sequence of any one of SEQ ID NOs: 22, 23, 24 and 25 or a variant amino acid sequence having no more than 1, 2, 3, 4, 5, 6, 7, 8, 9, or 10 amino acid additions, deletions, substitutions, or combinations thereof within the sequence of any one of SEQ ID NOs: 22, 23, 24 and 25, said addition, deletion, or substitution being outside of CDR1, CDR2 and CDR3 (underlined in the sequence for convenience), wherein SEQ ID NOs: 22, 23, 24 and 25 are as following

(SEQ ID NO: 22)
MAEVQLVESGGGLVQAGDSLRLSCAASGLTLSRYAMGWFRQAPGNEREF
VAVITASGRTTLYADSLKGRFTISRDNAKNTVALQMQSLKPEDTAVYYC
AADYGSGKYTRRQSEYDYWGQGTQVTVSSEPKTPKPQPAAA;
(SEQ ID NO: 23)
MAEVQLVESGGGLVQAGDSLRLSCATSGLTFSRYAMGWFRQAPGNEREF
VAVITASGRTTLYADSVKGRFTISRDNAKNTVALQMQSLKPEDTAVYYC
AADYGNKRYTRRQSEYEYWGQGTQVTVSSAAA;
(SEQ ID NO: 24)
MADVQLVESGGGLVQAGDSLRLSCAASGLTFSRYAMGWFRQAPGNEREF
VAVITASGRTTLYADSVKGRFTISRDNAKNTVALQMQSLKPEDTAVYYC
AADYGTSRYTRRQSEYEYWGQGTQVTVSSAAA;
and
(SEQ ID NO: 25)
MAEVQLQASGGGSVQPGGSLRLSCSASGLTFSRYAMGWFRQAPGNEREF
VAVITASGRTTLYADSVKGRFTISRDNAKNTVALQMQSLKPEDTAVYYC
AADYGGGRYTRRQSEYEYWGQGTQVTVSSAAA

In a particular aspect, the present invention relates to a bivalent molecule comprising two single domain antibodies directed against γ-H2AX as described herein. The two single domain antibodies can be the same or different.

Optionally, the bivalent molecule is a bivalent protein in which the two single domain antibodies are connected as a protein fusion. Optionally, the two single domain antibodies are connected via a peptide linker.

The linker is usually 3-44 amino acid residues in length. Preferably, the linker has 3-30 amino acid residues in length. Examples of linker sequences are Gly/Ser linkers of different length including (Gly₄Ser)₄, (Gly₄Ser)₃, (Gly₄Ser)₂, Gly₄Ser, Gly₃Ser, Gly3, Gly₂Ser and (Gly₃Ser₂)₃. In a particular aspect, the linker is (Gly₄Ser)₃.

In a particular aspect, the bivalent protein can comprise, essentially consist in or consist in an amino acid sequence selected from the group consisting of

(SEQ ID NO: 26)
MA(E/D)VQLXXSGGGXVQXG(G/D)SLRLSC(S/A)(A/T)SGLT(F/L)SRYAMGWFRQAPGNEREFVAVITASG
RTTLYADS(V/L)KGRFTISRDNAKNTVALQMQSLKPEDTAVYYCAADYGX₁X₂X₃YTRRQSEYX₄YWGQGTQV
TVSS(X)ₙAA (A/-)-linker
MA(E/D)VQLXXSGGGXVQXG(G/D)SLRLSC(S/A)(A/T)SGLT(F/L)SRYAMGWFRQAPGNEREFVAVITASG
RTTLYADS(V/L)KGRFTISRDNAKNTVALQMQSLKPEDTAVYYCAADYGX₁X₂X₃YTRRQSEYX₄YWGQGTQV
TVSS(X)ₙAAA,

wherein “linker” is a peptide linker, n is 0-10, preferably 0 or 9, more preferably 0; X is any amino acid; X₁ and X₂ are any amino acid, X₃ is K or R, and X₄ is D or E

(SEQ ID NO: 27)
MA(E/D)VQLXXSGGGXVQXG(G/D)SLRLSC(S/A)(A/T)SGLT(F/L)SRYAMGWFRQAPGNEREFVAVITASG
RTTLYADS(V/L)KGRFTISRDNAKNTVALQMQSLKPEDTAVYYCAADYGX₁X₂X₃YTRRQSEYX₄YWGQGTQV
TVSS(X)ₙAA (A/-)
GGGSGGGSGGGSMA(E/D)VQLXXSGGGXVQXG(G/D)SLRLSC(S/A)(A/T)SGLT(F/L)SRYAMGWFRQAP
GNEREFVAVITASGRTTLYADS(V/L)KGRFTISRDNAKNTVALQMQSLKPEDTAVYYCAADYGX₁X₂X₃YTRRQ
SEYX₄YWGQGTQVTVSS(X)ₙAAA,

wherein n is 0-10, preferably 0 or 9, more preferably 0; X is any amino acid; X₁ and X₂ are any amino acid, X₃ is K or R, and X₄ is D or E;

(SEQ ID NO: 28)
-MA(E/D)VQLVESGGGLVQAGDSLRLSCA(A/T)SGLTFSRYAMGWFRQAPGNEREFVAVITASGRTTLYADS
VKGRFTISRDNAKNTVALQMQSLKPEDTAVYYCAADYGX₁X₂RYTRRQSEYX₄YWGQGTQVTVSSAA (A/-)-
linker
MA(E/D)VQLVESGGGLVQAGDSLRLSCA(A/T)SGLTFSRYAMGWFRQAPGNEREFVAVITASGRTTLYADS
VKGRFTISRDNAKNTVALQMQSLKPEDTAVYYCAADYGX₁X₂RYTRRQSEYX₄YWGQGTQVTVSSAAA,

wherein “linker” is a peptide linker, and X₁ and X₂ are any amino acid and X₄ being D or E;

(SEQ ID NO: 29)
-MA(E/D)VQLVESGGGLVQAGDSLRLSCA(A/T)SGLTFSRYAMGWFRQAPGNEREFVAVITASGRTTLYADS
VKGRFTISRDNAKNTVALQMQSLKPEDTAVYYCAADYGX₁X₂RYTRRQSEYX₄YWGQGTQVTVSSAA (A/-)
GGGSGGGSGGGSMA(E/D)VQLVESGGGLVQAGDSLRLSCA(A/T)SGLTFSRYAMGWFRQAPGNEREFVA
VITASGRTTLYADSVKGRFTISRDNAKNTVALQMQSLKPEDTAVYYCAADYGX₁X₂RYTRRQSEYX₄YWGQGT
QVTVSSAAA,

with X₁ and X₂ being any amino acid and X₄ being D or E;

(SEQ ID NO: 30)
-MAEVQLVESGGGLVQAGDSLRLSCAASGLTLSRYAMGWFRQAPGNEREFVAVITASGRTTLYADSLKGRFTI
SRDNAKNTVALQMQSLKPEDTAVYYCAADYGSGKYTRRQSEYDYWGQGTQVTVSSEPKTPKPQPAA (A/-)-
linker-
MAEVQLVESGGGLVQAGDSLRLSCAASGLTLSRYAMGWFRQAPGNEREFVAVITASGRTTLYADSLKGRFTI
SRDNAKNTVALQMQSLKPEDTAVYYCAADYGSGKYTRRQSEYDYWGQGTQVTVSSEPKTPKPQPAAA,

wherein “linker” is a peptide linker;

(SEQ ID NO: 31)
MAEVQLVESGGGLVQAGDSLRLSCAASGLTLSRYAMGWFRQAPGNEREFV
AVITASGRTTLYADSLKGRFTISRDNAKNTVALQMQSLKPEDTAVYYCAA
DYGSGKYTRRQSEYDYWGQGTQVTVSSEPKTPKPQPAA (A/-) GGGSG
GGSGGGSMAEVQLVESGGGLVQAGDSLRLSCAASGLTLSRYAMGWFRQAP
GNEREFVAVITASGRTTLYADSLKGRFTISRDNAKNTVALQMQSLKPEDT
AVYYCAADYGSGKYTRRQSEYDYWGQGTQVTVSSEPKTPKPQPAAA;
(SEQ ID NO: 32)
MAEVQLVESGGGLVQAGDSLRLSCATSGLTFSRYAMGWFRQAPGNEREFV
AVITASGRTTLYADSVKGRFTISRDNAKNTVALQMQSLKPEDTAVYYCAA
DYGNKRYTRRQSEYEYWGQGTQVTVSSAA (A/-) - linker - MAE
VQLVESGGGLVQAGDSLRLSCATSGLTFSRYAMGWFRQAPGNEREFVAVI
TASGRTTLYADSVKGRFTISRDNAKNTVALQMQSLKPEDTAVYYCAADYG
NKRYTRRQSEYEYWGQGTQVTVSSAAA,

wherein “linker” is a peptide linker;

(SEQ ID NO: 33)
MAEVQLVESGGGLVQAGDSLRLSCATSGLTFSRYAMGWFRQAPGNEREFV
AVITASGRTTLYADSVKGRFTISRDNAKNTVALQMQSLKPEDTAVYYCAA
DYGNKRYTRRQSEYEYWGQGTQVTVSSAA (A/-) GGGSGGGSGGGSMA
EVQLVESGGGLVQAGDSLRLSCATSGLTFSRYAMGWFRQAPGNEREFVAV
ITASGRTTLYADSVKGRFTISRDNAKNTVALQMQSLKPEDTAVYYCAADY
GNKRYTRRQSEYEYWGQGTQVTVSSAAA;
(SEQ ID NO: 34)
MADVQLVESGGGLVQAGDSLRLSCAASGLTFSRYAMGWFRQAPGNEREFV
AVITASGRTTLYADSVKGRFTISRDNAKNTVALQMQSLKPEDTAVYYCAA
DYGTSRYTRRQSEYEYWGQGTQVTVSSAA (A/-) - linker - MAD
VQLVESGGGLVQAGDSLRLSCAASGLTFSRYAMGWFRQAPGNEREFVAVI
TASGRTTLYADSVKGRFTISRDNAKNTVALQMQSLKPEDTAVYYCAADYG
TSRYTRRQSEYEYWGQGTQVTVSSAAA,

wherein “linker” is a peptide linker;

(SEQ ID NO: 35)
MADVQLVESGGGLVQAGDSLRLSCAASGLTFSRYAMGWFRQAPGNEREFV
AVITASGRTTLYADSVKGRFTISRDNAKNTVALQMQSLKPEDTAVYYCAA
DYGTSRYTRRQSEYEYWGQGTQVTVSSAA (A/-) GGGSGGGSGGGSMA
DVQLVESGGGLVQAGDSLRLSCAASGLTFSRYAMGWFRQAPGNEREFVAV
ITASGRTTLYADSVKGRFTISRDNAKNTVALQMQSLKPEDTAVYYCAADY
GTSRYTRRQSEYEYWGQGTQVTVSSAAA;
(SEQ ID NO: 36)
MAEVQLQASGGGSVQPGGSLRLSCSASGLTFSRYAMGWFRQAPGNEREFV
AVITASGRTTLYADSVKGRFTISRDNAKNTVALQMQSLKPEDTAVYYCAA
DYGGGRYTRRQSEYEYWGQGTQVTVSSAA (A/-) - linker - MAE
VQLQASGGGSVQPGGSLRLSCSASGLTFSRYAMGWFRQAPGNEREFVAVI
TASGRTTLYADSVKGRFTISRDNAKNTVALQMQSLKPEDTAVYYCAADYG
GGRYTRRQSEYEYWGQGTQVTVSSAAA,

wherein “linker” is a peptide linker; and

(SEQ ID NO: 37)
MAEVQLQASGGGSVQPGGSLRLSCSASGLTFSRYAMGWFRQAPGNEREFV
AVITASGRTTLYADSVKGRFTISRDNAKNTVALQMQSLKPEDTAVYYCAA
DYGGGRYTRRQSEYEYWGQGTQVTVSSAA (A/-) GGGSGGGSGGGSMA
EVQLQASGGGSVQPGGSLRLSCSASGLTFSRYAMGWFRQAPGNEREFVAV
ITASGRTTLYADSVKGRFTISRDNAKNTVALQMQSLKPEDTAVYYCAADY
GGGRYTRRQSEYEYWGQGTQVTVSSAAA;

or a variant amino acid sequence having no more than 1, 2, 3, 4, 5, 6, 7, 8, 9, or 10 amino acid additions, deletions, substitutions, or combinations thereof, said addition, deletion, or substitution being outside of CDR1, CDR2 and CDR3 (underlined in the sequence for convenience). In a particular aspect, the (A/−) is no amino acid.

Is also contemplated herein a protein with higher valance than a bivalent protein. The present disclosure may relate to a protein (monomeric or polymeric) comprising 2, 3, 4, 5 or 6 single domain antibody as described herein.

Optionally, the single domain antibody or the bivalent molecule is labelled with a detectable entity (“label”). The term “label”, as used herein, refers to any atom or molecule that can be used to provide a quantifiable signal and that can be attached to a single domain antibody or bivalent molecule as disclosed herein via a covalent bond or a noncovalent interaction (e.g., through ionic or hydrogen bonding, or via immobilization, adsorption, or the like). A label may be selected from the group consisting in a radiolabel, an enzyme label, a fluorescent label, a bioluminescent molecule, a biotin-avidin label, a chemiluminescent label, and a detectable entity. Optionally, the detectable entity can be a tag that can be detected by an antibody specific for the tag.

Optionally, the detectable label is selected from the group consisting of: a hapten, a fluorescent dye, a fluorescent protein, a chromophore, a metal ion, a gold particle, a silver particle, a magnetic particle, a polypeptide, an enzyme, a luminescent compound, or an oligonucleotide.

In a preferred aspect, the detectable label is a fluorescent protein. For instance, the fluorescent protein can be selected in the non-exhaustive list comprising Green Fluorescent Protein, Enhanced Green Fluorescent Protein (EGFP), Enhanced Yellow Fluorescent Protein (EYFP), Venus, mVenus, Citrine, mCitrine, Cerulean, mCerulean, Orange Fluorescent Protein (OFP), mNeonGreen, moxNeonGreen, mCherry, mTagBFP, mTurquoise, mScarlet, mWasabi, mOrange, mStrawberry and dTomato.

In a very specific aspect, the fluorescent protein is dTomato and has the following sequence:

(SEQ ID NO: 38)
MVSKGEEVIKEFMRFKVRMEGSMNGHEFEIEGEGEGRPYEGTQTAKLKVT
KGGPLPFAWDILSPQFMYGSKAYVKHPADIPDYKKLSFPEGFKWERVMNF
EDGGLVTVTQDSSLQDGTLIYKVKMRGTNFPPDGPVMQKKTMGWEASTER
LYPRDGVLKGEIHQALKLKDGGHYLVEFKTIYMAKKPVQLPGYYYVDTKL
DITSHNEDYTIVEQYERSEGRHHLFL.

The detectable label can be a fluorescent dye, for instance selected in the non-exhaustive list including Oregon Green®, Pacific Blue™, Pacific Orange™, Pacific Green™, Cascade Blue™, Cascade Yellow™, Lucifer Yellow™, Marina Blue™, and Texas Red® (TxRed); an AlexaFluor®(AF) dye such as AF350, AF405, AF488, AF500, AF514, AF532, AF546, AF555, AF568, AF594, AF610, AF633, AF635, AF647, AF680, AF700, AF710, AF750, AF790, and AF800; a Cy dye such as Cy2, Cy3, Cy3.5, Cy5, Cy5.5, Cy7, and Cy 7.5; Atto 390, Atto 425, Atto 465, Atto 488, Atto 495, Atto 514Atto 520, Atto 532, Atto 550, Atto 565, Atto 590, Atto 594, Atto 610, Atto 620, Atto 633, Atto 647, Atto 655, Atto 665, Atto 680, Atto 700, Atto 725, Atto 740, Super Bright™ 436, Super Bright™ 600, Super Bright™ 645, Super Bright™ 702, Super Bright™ 780, Brilliant™ Violet 421, Brilliant™ Violet 480, Brilliant™ Violet 510, Brilliant™ Violet 605, Brilliant Violet™ 650, Brilliant Violet™ 711, Brilliant Violet™ 786, Brilliant™ Ultraviolet 395 (BUV395), Brilliant™ Ultraviolet 496 (BUV496), Brilliant™ Ultraviolet 563 (BUV563), Brilliant™ Ultraviolet 661 (BUV661), Brilliant™ Ultraviolet 737 (BUV737), Brilliant™ Ultraviolet 805 (BUV805), Brilliant™ Blue 515 (BB515), Brilliant™ Blue 700 (BB700) and IR Dye 680, IR Dye 680LT, IR Dye 700, IR Dye 700DX, IR Dye 800, IR Dye 800RS, and IR Dye 800CW.

The detectable label can be a hapten such as a fluorescein or a derivative thereof, fluorescein isothiocyanate, carboxyfluorescein, dichlorotriazinylamine fluorescein, digoxigenin, dinitrophenol (DNP), trinitrophenol (TNP), and biotin.

Optionally, the detectable molecule can be a detectable tag, preferably a peptide detectable tag. Non-limiting example of tag includes E6 tag (for instance of sequence TSMFQDPQERPRASA).

Alternatively, the detectable label can be a bioluminescent molecule or an enzyme such as luciferase, β-galactosidase, β-lactamase, peroxidase, alkaline phosphatase, β-glucuronidase, and β-glucosidase.

The detectable label can be a radiolabel, such as a radionuclide selected from the group consisting of: carbon (14C), chromium (5ICr), cobalt (57Co), fluorine (18F), gadolinium (I53Gd, I59Gd), germanium (68Ge), holmium (166H0), indium (1151h, 1131h, 1121h, min), iodine (1251, 1231, 1211), lanthanium (I40La), lutetium (I77Lu), manganese (54Mn), molybdenum (99 Mo), palladium (103 Pd), phosphorous (32 P), praseodymium (142 Pr), promethium (149Pm), rhenium (I86Re, I88Re), rhodium (I05Rh), rutheroium (97Ru), samarium (I53Sm), scandium (47Sc), selenium (75Se), (85Sr), sulphur (35S), technetium (99Tc), thallium (20ITi), tin (H3Sn, H7Sn), tritium (3H), xenon (I33Xe), ytterbium (I69Yb, I75Yb), and yttrium (90Y).

Accordingly, the present disclosure relates to a single domain antibody or bivalent protein as disclosed herein conjugated to a detectable label. The methods for preparing such a conjugate are well-known in the art. Optionally, the sequence of the single domain antibody or the bivalent protein can be modified by a substitution or addition of a residue suitable for the conjugation of the detectable label. Then, the amino acid sequence of the single domain antibody or bivalent protein includes a substitution or addition of a residue, preferably a cysteine, preferably near to the C-terminal end, for instance within the 2-10 most C-terminal amino acids of the single domain antibody or bivalent protein as described herein, more preferably within the 3-5 most C-terminal amino acids. For instance, the additional residue, preferably a cysteine residue, is added before the stretch of three A (i.e., AAA replaced by CAAA). This additional residue, preferably a cysteine residue, allows the introduction of a covalent link with a detectable label, in particular a fluorescent molecule, for instance by thiol maleimide reaction.

Optionally, the label is a protein and is fused or linked to the single domain antibody or the bivalent molecule, thereby forming a protein fusion. The label can be fused or linked at the N-terminal end of the single domain antibody or the bivalent molecule, or at the C-terminal end of the single domain antibody or the bivalent molecule or, in the context of the bivalent molecule, between the two single domain antibodies. In a particular aspect, the label is fused or linked at the C-terminal end of the single domain antibody or the bivalent molecule.

Optionally, the single domain antibody or the bivalent protein can further comprise tag sequence, such as a histidine tag, useful for the purification of the recombinant protein. Optionally, the single domain antibody or the bivalent protein can further comprise a NLS sequence (nuclear localization signal).

The present invention further relates to a nucleic acid sequence encoding the single domain antibody or the bivalent protein as disclosed above, an expression cassette comprising such a nucleic acid sequence, a vector comprising such a nucleic acid sequence or expression cassette, and a host cell comprising such a nucleic acid sequence, expression cassette or vector.

Optionally, the promoter used to control the expression of the single domain antibody or the bivalent protein is a weak promoter. Optionally, the expression vector is a low copy number vector. Optionally, the expression vector may comprise a restriction site allowing the insertion of a detectable label so as to obtain a protein fusion comprising the single domain antibody or the bivalent protein and the detectable protein.

The present disclosure also relates to a method for producing the single domain antibody or the bivalent protein as described herein comprising expressing the single domain antibody or the bivalent protein in a host cell and recovering the produced single domain antibody or bivalent protein.

The present disclosure relates to the single domain antibody or the bivalent protein or a nucleic acid, expression cassette or vector encoding it as a research tool. For instance, it relates to a kit comprising the single domain antibody or the bivalent protein as described herein or an expression vector encoding the single domain antibody or the bivalent protein and a leaflet for the use of this reagent. Preferably, the single domain antibody or the bivalent protein comprises a detectable label as detailed above.

The present disclosure relates to the use of the single domain antibody or the bivalent protein as described herein or a nucleic acid, expression cassette or vector encoding it for detecting and/or quantifying and/or monitoring γ-H2AX in a cell or a cellular extract thereof, especially γ-H2AX foci. It relates to the use of the single domain antibody or the bivalent protein as described herein or a nucleic acid, expression cassette or vector encoding it for detecting or monitoring DNA damage or Replication stress in a cell or a cellular extract thereof. The use is a non-therapeutic use. The use can be an in vitro use, an in cellulo use or an ex vivo use (on isolated cells). In particular, the in vivo use can be excluded.

Optionally, the single domain antibody or bivalent protein is used in one of the following assays: ELISA, flow cytometry, immunofluorescence, live cell imaging (non fixed), immunoprecipitation, in particular Chromatin immunoprecipitation, and Western blot.

The present disclosure further relates to a method for detecting and/or quantifying and/or monitoring γ-H2AX in a cell, comprising contacting the cell with a single domain antibody or a bivalent protein as described herein or with a nucleic acid, expression cassette or vector encoding said single domain antibody or bivalent protein, and detecting and/or quantifying and/or monitoring the single domain antibody or bivalent protein in the cell or a cellular extract thereof. The method can be for detecting or quantifying or monitoring DNA damage or Replication stress in a cell. The method is a non-therapeutic method. The method can be an in vitro method, an in cellulo method or an ex vivo method (on isolated cells). In particular, the in vivo method can be excluded. The method allows to study the dynamics of the chromosome modification in an individual cell.

Optionally, the cell is a cancer cell. Optionally, the cell is a living cell. Optionally, the cell is a fixed cell. Preferably, the cell is an eukaryotic cell, more preferably a mammalian cell.

Optionally, the cell is contacted or has been contacted or will be contacted with a test compound or molecule simultaneously or before the contacting step with the single domain antibody or bivalent protein. The test compound or molecule can be any compound or molecule, especially can be a compound or molecule known or suspected to be a genotoxic agent. Optionally, the use and method as disclosed above is preferable after induction of DNA damage or replication stress.

The present disclosure may relate to the use of the single domain antibody or the bivalent protein as described herein or a nucleic acid, expression cassette or vector encoding it for screening or identifying a compound or a molecule having a genotoxic effect; or to a method for screening or identifying a compound or a molecule having a genotoxic effect, the method comprising contacting a eukaryotic cell with a compound or a molecule, the cell expressing the single domain antibody or the bivalent protein as described herein or the cell being contacted with the single domain antibody or the bivalent protein as described herein, and detecting and/or quantifying and/or monitoring the single domain antibody or the bivalent protein in the cell, thereby determining the effect of the compound or molecule on DNA damage or replication stress or determining the genotoxic effect of the compound or molecule. In one aspect, the compound or molecule is selected if no genotoxic effect is detected. In an alternative aspect, the compound or molecule is selected if a genotoxic effect is detected. Optionally, the effect observed for the compound or molecule can be compared with one or several compounds or molecules of reference for which the genotoxic effect or the absence of genotoxic effect is well-documented.

Preferably, the single domain antibody or the bivalent protein is detected, quantified or monitored in the nucleus of the cell. Preferably, the single domain antibody or the bivalent protein is use for detecting, quantifying or monitoring the γ-H2AX foci.

Preferably, the single domain antibody or the bivalent protein is linked to a fluorescent label as detailed above and the single domain antibody or the bivalent protein is detected, quantified or monitored by the fluorescence of the fluorescent label. The advantage is that the detection, quantification or monitoring can be carried in a one-step process.

Optionally, the single domain antibody or the bivalent protein is monitored for a period of time, for instance by video recording, to follow the event occurring in the living cell after induction of DNA damage or replication stress.

Optionally, other kind of detectable label can be used and the method may comprise the detection of the detectable label through the addition or the use of a mean specific for the detectable label. For instance, if the detectable label is a tag, an antibody specific for this tag can be used to detect the detectable label.

### Definition

As used herein, the term “H2AX” refers to H2A histone family member X (H2AX). It is described in UniProtKB under reference P16104 for human and P27661 for mouse. Human sequence of H2AX is the following

MSGRGKTGGKARAKAKSRSSRAGLQFPVGRVHRLLRKGHYAERVGAGAPV
YLAAVLEYLTAEILELAGNAARDNKKTRIIPRHLQLAIRNDEELNKLLGG
VTIAQGGVLPNIQAVLLPKKTSATVGPKAPSGGKKATQASQEY.

Mouse sequence of H2AX is the following

MSGRGKTGGKARAKAKSRSSRAGLQFPVGRVHRLLRKGHYAERVGAGAPV
YLAAVLEYLTAEILELAGNAARDNKKTRIIPRHLQLAIRNDEELNKLLGG
VTIAQGGVLPNIQAVLLPKKSSATVGPKAPAVGKKASQASQEY.

When the serine residue is phosphorylated, the protein is called gammaH2AX or γ-H2AX.

As used herein, the terms “antibody” refers to immunoglobulin molecules and immunologically active portions of immunoglobulin molecules, i.e., molecules that contain an antigen-binding site that specifically binds an antigen. As such, the term antibody encompasses not only whole antibody molecules, but also antigen-binding antibody fragments as well as variants (including derivatives) of antibodies and antibody fragments. In particular, the term antibody refers to heavy-chain only antibodies, VHH, fragments and derivatives thereof such (VHH)₂ fragments and single domain antibodies.

As used herein, the terms “Heavy-chain only antibody” or “HCAbs” refer to immunoglobulins which are devoid of light chains and consist in two heavy chains. These antibodies do not rely upon the association of heavy and light chain variable domains for the formation of the antigen-binding site but instead the variable domain of the heavy polypeptide chains alone naturally forms the complete antigen binding site. Each heavy chain comprises a constant region and a variable domain which enables the binding to a specific antigen, epitope or ligand. As used herein, HCAbs encompass heavy chain antibodies of the camelid-type in which each heavy chain comprises a variable domain called VHH and two constant domains. Such heavy-chain antibodies directed against a specific antigen can be obtained from immunized camelids. Camelids encompass dromedary, camel, lama and alpaca. Camelid HCAbs have been described by Hamers-Casterman et al., Nature, 1993, 363:446. Other examples of HCAb are immunoglobulin-like structures (Ig-NAR) from cartilaginous fishes. Heavy-chain antibodies can be humanized using well-known methods.

The terms “single domain antibody”, “sdAb” and “nanobody” are used interchangeably and have the same meaning. As used herein, the term single domain antibody refers to a single variable domain derived from a heavy chain antibody, which is able to bind an antigen, an epitope or a ligand alone, that is to say, without the requirement of another binding domain. A single domain antibody may be or may derive from VHH and V-NAR. V-NAR refers to the variable domain found in immunoglobulin-like structures (Ig-NAR) discovered in cartilaginous fishes such as sharks. For review about single domain antibodies, one may refer to Saerens et al., Current Opinion in Pharmacology, 2008, 8:600-608, the disclosure of which being incorporated by reference. In a preferred aspect, the single domain antibody according to the present disclosure is a synthetic single domain antibody. As used herein, the term “synthetic” means that such antibody has not been obtained from fragments of naturally occurring antibodies but produced from recombinant nucleic acids comprising artificial coding sequences (cf. WO 2015/063331).

The term “VHH”, as used herein, refers to an antibody fragment consisting of the VH domain of camelid heavy-chain antibody. VHH fragments can be produced through recombinant DNA technology in a number of microbial hosts (bacterial, yeast, mould), as described in WO 94/29457. Alternatively, binding domains can be obtained by modification of the VH fragments of classical antibodies by a procedure termed “camelization”, described by Davies et al, 1995. Dimers of VHH fragments, i.e. (VHH)₂, can be generated by fusing two sequences encoding VHH fragments, end to end, e.g., by PCR. Preferably, the (VHH)₂ fragment is monospecific.

The variable domain of an antibody of the present disclosure comprises at least three complementarity determining region (CDR) which determine its binding specificity. Preferably, in a variable domain, the CDRs are distributed between framework regions (FRs). The variable domain thus contains at least 4 framework regions interspaced by 3 CDR regions, resulting in the following typical antibody variable domain structure: FR1-CDR1-FR2-CDR2-FR3-CDR3-FR4. CDRs and/or FRs of the single domain antibody of the present disclosure may be fragments or derivatives from a naturally-occurring antibody variable domain or may be synthetic.

As used herein, the terms “Amino acid modification”, “amino acid change”, and “mutation” are used interchangeably and refer to a change in an amino acid sequence such as a substitution, an insertion, and/or a deletion. By “amino acid substitution” or “substitution” herein is meant the replacement of an amino acid at a particular position in a parent amino acid sequence with another amino acid. By “amino acid insertion” or “insertion” is meant the addition of an amino acid at a particular position in a parent amino acid sequence. By “amino acid deletion” or “deletion” is meant the removal of an amino acid at a particular position in a parent amino acid sequence.

The amino acid substitutions may be conservative. A conservative substitution is the replacement of a given amino acid residue by another residue having a side chain (“R-group”) with similar chemical properties (e.g., charge, bulk and/or hydrophobicity). In general, a conservative amino acid substitution will not substantially change the functional properties of a protein. Conservative substitutions and the corresponding rules are well-described in the state of the art. For instance, conservative substitutions can be defined by substitutions within the groups of amino acids reflected in the following tables:

As used herein, the term “fusion protein” or “protein fusion” are equivalent and refers to protein created through the joining of two or more genes that originally coded for separate proteins. Translation of this fusion gene results in a single polypeptide with functional properties derived from each of the original proteins. Preferably, the fusion protein of the invention is a recombinant fusion protein created artificially by recombinant DNA technology.

TABLE A
Amino Acid Residue

| Amino Acid groups | Amino Acid Residues |
|---|---|
| Acidic Residues | ASP and GLU |
| Basic Residues | LYS, ARG, and HIS |
| Hydrophilic Uncharged Residues | SER, THR, ASN, and GLN |
| Aliphatic Uncharged Residues | GLY, ALA, VAL, LEU, and ILE |
| Non-polar Uncharged Residues | CYS, MET, and PRO |
| Aromatic Residues | PHE, TYR, and TRP |

TABLE B
Alternative Conservative Amino Acid Residue Substitution Groups

| | | | |
|---|---|---|---|
| 1 | Alanine (A) | Serine (S) | Threonine (T) |
| 2 | Aspartic acid (D) | Glutamic acid (E) | |
| 3 | Asparagine (N) | Glutamine (Q) | |
| 4 | Arginine (R) | Lysine (K) | |
| 5 | Isoleucine (I) | Leucine (L) | Methionine (M) |
| 6 | Phenylalanine (F) | Tyrosine (Y) | Tryptophan (W) |

TABLE C
Further Alternative Physical and Functional
Classifications of Amino Acid Residues

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, |
| | W, and Y |
| Negatively charged residues | D and E |
| Polar residues | C, D, E, H, K, N, Q, R, S, and T |
| Small residues | A, C, D, G, N, P, S, T, and V |
| Very small residues | A, G, and S |
| Residues involved in turn formation | A, C, D, E, G, H, K, N, Q, R, S, |
| | P, and T |
| Flexible residues | E, Q, T, K, S, G, P, D, E, and R |

As used herein, (AA1/AA2) refers to the choice between the residue AA1 or the residue AA2. For instance, (E/D) means E or D; (A/T) means A or T; (G/D) means G or D; (S/A) means S or A; (F/L) means F or L; (V/L) means V or L; (A/−) means A or no amino acid.

As used herein, the term “consists essentially in” is intended to refer to an amino acid sequence that differs from that of a parent amino acid sequence by virtue of 1, 2, or 3 substitutions, additions, deletions or combination thereof.

As used herein, the terms “variant amino acid sequence”, variant polypeptide” or “variant” are equivalent and refer to an amino acid sequence that differs from that of a parent amino acid sequence by virtue of at least one amino acid modification. In the context of the invention, a variant is a variant of a variable domain, a CDR or a FR. Typically, a variant comprises from 1 to 40 amino acid modifications, preferably from 1 to 30 amino acid modifications, more preferably 1 to 20 amino acid modifications. In particular, the variant may have from 1 to 15 amino acid changes, e.g., 1, 2, 3, 4, 5, 6, 7, 8, 9, 10, 11, 12, 13, 14, 15 amino acid changes as compared to its parent amino acid sequence. In a specific aspect, the variant may have from 1 to 3 amino acid changes, e.g., 1, 2, or 3 amino acid changes as compared to its parent amino acid sequence. The variants may comprise one or several amino acid substitutions, and/or, one or several amino acid insertions, and/or one or several amino acid deletions. In some embodiments, the variant may comprise one or several conservative substitutions, e.g., as shown here above. In some other embodiments, the variant comprises one or several amino acid modifications in the framework domains.

As used herein, the term “expression cassette” refers to a nucleic acid construction comprising a coding region and regulatory regions necessary for expression, operably linked to the coding region. The expression “operably linked” indicates that the elements are combined in such a way that the expression of the coding region is under the control of the regulatory regions. Typically, a regulatory region is located upstream of the coding region at a distance compatible with the control of its expression. The regulatory region can include promoters, enhancers, silencers, attenuators, and internal ribosome entry sites (IRES). Spacer sequences may also be present between regulatory elements and the coding region, as long as they don't prevent its expression. An expression cassette may also include a start codon in front of a protein-encoding gene, splicing signals for introns, and stop codons, transcription terminators, polyadenylation sequences.

As used herein, the terms “promoter” and “transcriptional promoter” are equivalent and refer to a region of DNA that is part of the regulatory region of an expression cassette. The promoter is the regulatory element that initiates the transcription of a particular gene. Promoters are located near the transcription start site of genes, on the same strand and upstream on the DNA (towards the 5′ region of the sense strand).

As used herein, the term “expression vector” refers to a vector designed for gene expression in cells. An expression vector allows to introduce a specific gene into a target cell, and can commandeer the cell's mechanism for protein synthesis to produce the protein encoded by the gene. An expression vector comprises expression elements including, for example, a promoter, the correct translation initiation sequence such as a ribosomal binding site and a start codon, a termination codon, and a transcription termination sequence. An expression vector may also comprise other regulatory regions such as enhancers, silencers and boundary elements/insulators to direct the level of transcription of a given gene. The expression vector can be a vector for stable or transient expression of a gene.

### BRIEF DESCRIPTION OF THE FIGURES

FIG. 1: Development and selection of specific anti-γ-H2AX nanobodies.

FIG. 1A is a schematic representation of a phage display selection round (left). The histogram on the right shows the number of phages retained on plate after 2 rounds of selection with 3 different libraries issued from peripheral blood mononuclear cells (PBMCs) of individual animals.

FIG. 1B shows the specific binding capacity of the phages selected from the library 2 were assayed by phage-ELISA with either peptides as indicated (left) or histones extracted from H-treated (treatment with hydroxyurea) or untreated (NT) cells (right), both immobilized on plate.

FIG. 1C shows four individual VHH-phages (VHH: variable domain) identified by sequencing (A4, A9, C6 and G2) subjected to phage-ELISA. Their specific binding to either the non-phosphorylated (peptide) or the phosphorylated (phospho-peptide) H2AX C-terminal peptide is shown. Bound phages were revealed with an HRP-labelled anti-M13 conjugate (Materials and Methods).

FIG. 1D: the sequences of clones A4, A9, C6 and G2 are aligned based on homology, according to the Kabat numbering system. The residues highlighted correspond to the complementary-determining region (CDR) residues. Residues 114 to 125 are part of the hinge region. The R residues at positions 100C and 100D are shown in bold and the R residue indicated with an arrow is a hallmark residue of the VHH variable domain.

FIG. 2: Biochemical and structural analysis of the selected nanobodies.

FIG. 2A is an SDS-PAGE analysis of the purified nanobodies A9 and C6.

FIG. 2B shows the binding capacity of the purified samples shown in FIG. 2A which was tested by ELISA with either phosphorylated (phospho-peptide; 1 pg/mL) or non-phosphorylated (peptide; 1 μg/mL) C-terminal H2AX peptide coated on plate.

FIG. 2C is an immunofluorescence assay with the C6 nanobody. H1299 cells were treated for 24 hours with the indicated drugs (hydroxyurea, (H); or a combination of gemcitabine and a Chk-1 inhibitor (G+A)) and incubated after fixation with nanobody C6. Bound molecules were revealed with anti-tag E6 and Alexa 568-labelled anti-mouse IgG. The nuclei were counterstained with DAPI (4′,6-diamidino-2-phenylindole). Scale bar: 20 μm.

FIG. 2D is a quantification of the γ-H2AX fluorescence signal recorded following incubation of the cells treated as in FIG. 2C with either A9 or C6 nanobody.

FIG. 2E is a crystallographic 3D-structure of the C6 nanobody in complex with the phosphorylated peptide corresponding to γ-H2AX C-terminal tail (ApSQEY). The CDR1, CDR2 and CDR3 loops are respectively shown with arrows. The γ-H2AX tail is shown with an arrow and peptide residues are boxed.

FIGS. 2F and 2G are close-up view of the γ-H2AX tail peptide in the nanobody binding site. Residues are labelled as in FIG. 2E. Water molecules in the interface between the γ-H2AX tail and the nanobody are represented as spheres and hydrogen bonds are represented as dotted lines.

FIG. 3: C6 nanobody localization to the nucleus in drug-treated H1299 cells.

FIG. 3A is an immunofluorescence analysis of H1299 cells after transfection with the plasmid encoding the C6-mCherry chromobody. 24 hours post-transfection, the indicated drugs were added. After incubation for 24 hours, the cells were either fixed (−CSK buffer) or washed with cytoskeletal (CSK) buffer prior to fixation (+CSK buffer). The nuclei were counterstained with DAPI. The panel shows representative images recorded under the microscope (left) and the percentage of fluorescent cells observed in each condition is shown (right). Cut-off for negative cells was set on non-transfected cells using the maximal recorded value. Scale bar: 50 μm.

FIG. 3B: following transduction with either C6 nanobody or Fab 3F4, H1299 cells were treated with H and analyzed by immunofluorescence 48 hours post-treatment. Representative images are shown on the left. Bound nanobody or Fab were revealed with anti-E6T antibodies and secondary Alexa 568-labelled anti-mouse globulins. Scale bar: 20 μm. The quantification of the γ-H2AX mean fluorescence intensity (FI) of the monitored cells is shown on the right. The numbers indicated in brackets correspond to the number of cells analyzed in each condition.

FIG. 4: Binding performance of the bivalent C6 nanobody.

FIG. 4A is a schematic representation of the constructs used to produce bivalent nanobodies in E. coli cells. The four R residues of bivalent C6 nanobody (C6B) that have been altered to generate the mutant bivalent C6 nanobody (C6BM) are indicated.

FIG. 4B is an analysis by surface plasmon resonance (SPR) of the interaction of monovalent (C6; 180 nM) or bivalent (C6B; 80 nM) C6 nanobody with the phospho-peptide immobilized on chip. The curves show typical normalized profiles of the fractional occupancy calculated with the signals recorded for each nanobody (Materials and Methods). Injection of nanobody was stopped at the 120 seconds time-point and dissociation was analyzed during 700 seconds.

FIG. 4C is a representative immunofluorescence images of H- or G+A-treated H1299 cells following fixation and incubation with bivalent C6 nanobody (left). Bound material was revealed as described in the legend of FIG. 3. The nuclei were counterstained with DAPI. Scale bar: 20 μm. The quantification of the mean γ-H2AX fluorescence intensity (FI) of these analyzed cells and those monitored after incubation with the C6BM molecules is shown on the right. The number of cells analyzed in each condition is indicated.

FIG. 4D shows the detection of γ-H2AX in drug-treated H1299 cells with the fluorescently-labelled C6B. A depiction of the bivalent nanobody chemically conjugated to Alexa 568 is shown on the left. An immunofluorescence analysis of drug-treated H1299 cells after incubation with the labelled conjugate is shown on the right. Nuclei were counterstained with DAPI. Scale bar: 20 μm.

FIG. 4E is a box plot representation as in FIG. 4C of the normalized γ-H2AX fluorescence intensity detected with the C6B-Alexa 568 conjugate of H1299 cells after treatment with the indicated drugs or drug combinations. The data shown correspond to those recorded after log transformation. The full name and the concentration of the drugs used is indicated in the Materials and Methods section. The numbers indicated in the x axis correspond to the number of cells analyzed in each condition. NT, non-treated cells.

FIG. 4F shows a comparison of the C6B-Alexa 568 conjugate with the mAb 3F4 for detecting γ-H2AX in drug-treated H1299 cells. The FI data obtained with cells treated as in FIG. 4E and incubated with mAb 3F4 and Alexa 568-labelled secondary antibodies were plotted against the data shown in FIG. 4E. The means in each case were taken to generate the curve. The calculated Pearson correlation coefficient is indicated.

FIG. 5: Detection of γ-H2AX with the bivalent nanobody upon delivery by electroporation.

FIG. 5A: H1299 cells transduced with either C6B or C6BM nanobodies were treated with H and revealed with anti-tag E6 antibody and Alexa 568-labelled anti-mouse globulins 40 hours post-treatment. Typical immunofluorescence images of C6B-transduced cells taken with a confocal microscope after DAPI counterstaining are shown (lower images). Scale bar: 10 μm.

FIG. 5B: the quantification of the mean FI of cells transduced as in FIG. 5A with either C6B or C6BM are represented. The number of analyzed cells in each condition is indicated (bottom).

FIG. 5C: schematic representation of the C6B-mCherry (C6B-mCh) and the C6B-dTomato (C6B-dTo) fusion proteins used in the study.

FIG. 5D: analysis by SDS-PAGE of the purified C6B-mCherry (1) and the C6B-dTomato (2) fusion proteins. The proteolytic products observed in the C6B-mCherry samples are indicated with arrows. M, molecular weight markers (kDa).

FIG. 5E: analysis by immunofluorescence microscopy of the C6B-dTomato (C6B-dTo) and C6BM-dTomato (C6BM-dTo) fusion proteins following transduction in H1299 cells. 24 hours post-transduction, the cells were treated with H or left untreated (NT). The images show typical fields observed in each case under the microscope after fixation and DAPI counterstaining (lower images). Scale bar: 20 μm. An enlargement of one cell present in the field of the C6B-dTo samples following overlay of the red and blue channels with Fiji is shown below the original images.

FIG. 5F: the quantification of the nuclear mean FI of C6B-dTo-transduced H1299 cells after treatment with the indicated drugs is shown. The numbers at the bottom correspond to the number of cells analyzed in each case.

FIG. 6: Visualization of the binding of the bivalent nanobody in live H1299 cells and analysis of its effect after pulse treatment with hydroxyurea.

FIG. 6A: representative wide-field fluorescence microscopy images of H1299 cells transduced with the C6B-dTomato fusion protein and subsequently treated with the indicated drugs or left untreated (NT). Images with an identical exposure time were taken 24 hours after treatment. The amount of protein used for the transduction in each case is also indicated. Scale bar: 10 μm. The nucleus shown in the inset correspond to the nucleus of the NT panel after 8-fold enhancement of the exposure time.

FIG. 6B: analysis of the movement of the foci formed in C6B-dTo-transduced H1299 cells treated with H. 24 hours post-treatment, the cells were analyzed as in FIG. 6A and pictures were taken every minute (total time: 10 minutes). The recorded images were processed as indicated in Materials and Methods section and show the trajectories of the foci present in two typical cells after 0, 1, 5 and 10 minutes of incubation. Scale bar: 10 μm.

FIG. 6C: growth rate of the transduced H1299 following pulse-treatment with H. After transduction with the indicated proteins, the cells were seeded on plate and pulse-treated during 24 hours with H. The curves correspond the number of cells after counting at 24, 48, 72 and 96 hours after seeding. The data correspond to the calculated ratios (number of cells in each case/number of cells at seeding time (0 h)).

FIG. 6D: variation of γ-H2AX levels in H1299 cells transduced with either PBS or the C6B or C6B-dTo proteins and treated for 24 hours (pulse treatment) with H as probed by Western blotting with mAb 3F4. Following treatment, the transduced cells (5×10⁵) were incubated in fresh medium and extracts (50 μg) were prepared at the indicated time points. β-actin was used as a loading control.

FIG. 7: Characterization of the A9 nanobody.

FIG. 7A: analysis by SDS-PAGE of the bacterially-expressed nanobodies. The gel shows the protein content of similar amounts of total (E), soluble (S) and insoluble (I) fractions of extracts obtained after lysis of the induced bacteria. The bands corresponding to the nanobody polypeptides are indicated with arrows.

FIGS. 7B and 7C: representative immunofluorescence images of drug-treated H1299 cells recorded after incubation with either A9 nanobody (FIG. 7B) or mAb 3F4 (FIG. 7C). Scale bar: 20 μm.

FIG. 7D: quantification of the signal obtained with the cells shown in FIG. 7C. The number of analyzed cells is indicated in brackets.

FIG. 8: Microscopic analysis of C6 and A9 nanobodies upon transfection.

FIG. 8A: representative confocal microscopy images of H1299 cells after transfection of the C6 nanobody-mCherry construct. The cells were treated as indicated in the legend of FIG. 2C. Scale bar: 20 μm.

FIGS. 8B and 8C: immunofluorescence analysis of H1299 cells after transfection with chromobody A9-GFP. The cells were treated as indicated in the legend of FIG. 3A. Representative images recorded under the microscope (FIG. 8B) and the corresponding percentage of fluorescent cells observed in each condition (FIG. 8C) are shown. Cut-off for negative cells was set on non-transfected cells using the maximal recorded value. Scale bar: 50 μm.

FIG. 9: Biochemical and fluorescence microscopic analyses of C6B an C6BM nanobodies.

FIG. 9A: purification and analysis on SDS gel of the C6B and C6BM nanobodies. Aliquots of affinity-purified protein samples (1 to 5 μg) were subjected to SDS-PAGE and subsequent Coomassie blue staining.

FIG. 9B: varying concentrations of C6B and C6BM nanobodies were probed by ELISA with fixed phospho-peptide on plate (0.1 μg/mL).

FIG. 9C: typical binding profiles of the C6 nanobody to the phospho-peptide as probed by SPR (Materials and Methods). The experimental values of each experiment are indicated.

FIG. 9D: immunofluorescence assay with the C6B nanobody in U2OS cells. Bound nanobodies were revealed with anti-E6 tag antibodies and Alexa Fluor 488 anti-mouse immunoglobulins. The nuclei were counterstained with DAPI (lower images). Scale bar: 20 μm.

FIG. 9E: representative immunofluorescence images of H- or G+A-treated H1299 cells following fixation and incubation with 2 ng/ml or 5 ng/ml of bivalent C6B nanobody bound material was revealed with anti-E6T antibody and Alexa 568-labelled anti-mouse globulins. The nuclei were counterstained with DAPI (lower images). Scale bar: 20 μm.

FIG. 10: Expression of the bivalent chromobodies in transfected cells.

FIG. 10A: representative immunofluorescence images of H1299 cells transfected with the pβA-C6B-E6T-mCherry construct. The transfected cells were treated with the indicated drugs during 24 hours and after cell fixation, expressed nanobody-mCherry fusions were monitored under a confocal microscope. Scale bar: 20 μm.

FIG. 10B: evaluation of the binding stability of the C6B-mCherry or the C6BM-mCherry fusions expressed in H1299 cells treated with the indicated drugs after transfection. The histograms show the percentage of fluorescent nuclei detected after fixation (−CSK buffer) or after a wash with CSK buffer prior to fixation (+CSK buffer). Up to 300 nuclei recorded from 3 independent experiments in each condition were analyzed to calculate the percentages.

FIG. 10C: representative immunofluorescence images of the transfected H1299 cells used in FIG. 10B. Scale bar: 50 μm.

FIG. 11: Transduction of the C6B or C6BM nanobodies in cancer cells.

FIG. 11A: transduction of C6B and C6BM in H1299 cells. Representative images recorded by fluorescence microscopy after treatment of the cells with H or left untreated (NT). The nanobodies were revealed as indicated in the legend of FIG. 4. Scale bar: 20 μm.

FIG. 11B: transduction of C6B nanobodies in U2OS cells treated as in FIG. 11A. Representative images taken with a confocal microscope after DAPI counterstaining (lower images) are shown. The C6B molecules were revealed as indicated in FIG. 9D. Scale bar: 10 μm.

FIG. 12: Specific detection of foci with the C6B-dTo chromobody upon transduction

FIG. 12A: comparison of the binding performance of C6-dTo and the C6B-dTo chromobodies. Equivalent amounts of monovalent or divalent chromobodies were delivered in H1299 cells and images were taken after treatment of the transduced cells for 24 hours with H. The pictures shown correspond to composite images of the dTomato and the DAPI signals. Scale bar: 20 μm.

FIG. 12B: H1299 cells were transduced with the C6B-dTo chromobody and treated as indicated in FIG. 12A. Prior to analysis by immunofluorescence microscopy, they were incubated with mAb 3F4 and Alexa 468-labelled secondary anti-mouse globulins. The pictures show the foci pattern of a typical cell following analysis with red (left) or the green (middle) filters. Stronger lightness shows the colocalization of the chromobody and the mAb at foci (right). Scale bar: 10 μm.

FIG. 12C: transduction of C6B-dTo chromobodies in U20S cells treated as in Fi.12A. Scale bar: 20 μm.

FIG. 12D: detection of foci in H1299 cells transduced with C6B-dTo chromobody and subsequent treatment with either clofarabine (C) or triapine (T). Typical nuclei after analysis as in FIG. 12A are shown. Scale bar: 10 μm.

FIG. 13: Visualization of the movement of the C6B-dTomato molecules in live H1299 cells and schematic representation of their binding in drug-injured cells.

FIG. 13A: two representative wide-field fluorescence microscopy images of H1299 cells transduced with the C6B-dTomato fusion protein and subsequently treated with G+A during 4 hours (left) were analyzed as indicated in the legend of FIG. 6. The trajectories of the γ-H2AX foci over a period of 10 minutes are shown. Scale bar: 10 μm.

FIG. 13B: the left panel represents the internalization and nuclear transport of the C6B-dTo molecules. Upon delivery in the cytoplasm by electroporation they bind to newly-synthesized nuclear proteins (square) and are piggybacked in the nucleus (right lower corner compartment). The accumulation of the C6B-dTo molecules at discrete sites upon treatment with H and γ-H2AX foci formation in the nucleus is shown in the right panel. Without drug treatment, the C6B-dTo molecules remain homogeneously distributed in the nucleus and the faint speckled staining observed after transduction is almost no more visible upon cell division.

### EXAMPLES

### Example 1: Development and Selection of Specific Anti-γ-H2AX Nanobodies by Phage Display

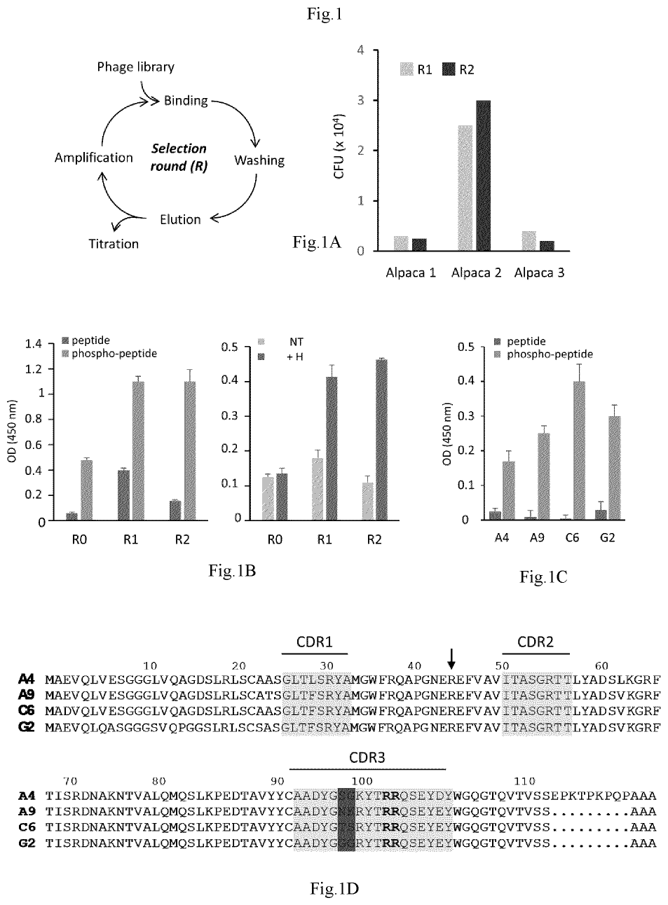

To produce a nanobody to track γ-H2AX in cells under DNA replication stress (RS), the inventors immunized alpacas with the phosphorylated peptide CKATQA(p) SQEY corresponding to the C-terminal end of γ-H2AX (residues 134-142). This peptide has been used in a previous study to generate monoclonal antibodies that are suitable for detecting γ-H2AX in various immunoassays (Moeglin, E. et al.; Cancers 2019, 11, 355, doi:10.3390/cancers11030355). Following immunization, the PBMCs were collected and VHH libraries of approximately 10⁷ independent clones were constructed. The phage display technology, which consist in displaying the VHH molecules on the tip of M13-based phages, allows selecting those that bind to the phospho-peptide immobilized on plate. This method of antigen display was preferred to other methods such as immobilization on magnetic beads since it previously allowed successful screening of cell culture supernatants containing monoclonal antibodies. Colony counting following the first round of panning (R1) showed that phages expressing a VHH against the phospho-peptide were only present in the repertoire of one animal (alpaca 2) (FIG. 1A). The same results were obtained after the second round of selection, indicating that only a low fraction of the cloned VHH molecules bound to the antigen. This suggests that immunization with the phospho-peptide did not trigger a strong heavy chain-only antibody response in the animals, but promoted mainly the synthesis of IgG1, since the sera collected from the 3 animals were positive when tested by ELISA with anti-alpaca immunoglobulins. Importantly, anti-γ-H2AX heavy chain-only antibodies could not be detected in the sera by immunoprecipitation with peptide-coated beads.

The inventors tested the binding specificity of the VHH-displaying phages selected from library 2 by comparing their reactivity against the phosphorylated and non-phosphorylated peptides coated on plate (phage-ELISA). The selected populations bound preferentially to the phospho-peptide, which suggests that the phosphate group at 5139 is important for the recognition (FIG. 1B, left). Interestingly, the inventors also observed that phages corresponding to the library without selection (R0) were able to react with the phospho-peptide, albeit to a lesser extent (FIG. 1B). Similar results were obtained when the same experiment was performed with nuclear extracts of U2OS cells treated for 24 hours with hydroxyurea (H), which induces RS and causes H2AX phosphorylation (FIG. 1B, right). This suggests that the VHH molecules displayed on the surface of the selected phages recognize γ-H2AX.

Individual clones of the positive phage population (R1 and R2 from library 2) were amplified and subjected to DNA sequencing. The alignment of 165 different sequences showed that almost all analyzed VHH clones have a similar amino acid composition, except at positions 98 and 99 (Kabat numbering) in the complementary-determining region 3 (CDR3) (FIG. 1D), meaning that they all may have arisen from a single B cell. The specific binding of the four most-represented variants (A4, A9, C6 and G2) to the phospho-peptide was confirmed by phage-ELISA (FIG. 1C). Collectively, these results show that the screening by phage display of several immune VHH libraries led to the selection of a unique VHH scaffold that specifically binds to γ-H2AX.

### Example 2: The Selected Nanobodies are Soluble in the Bacterial Cytoplasm

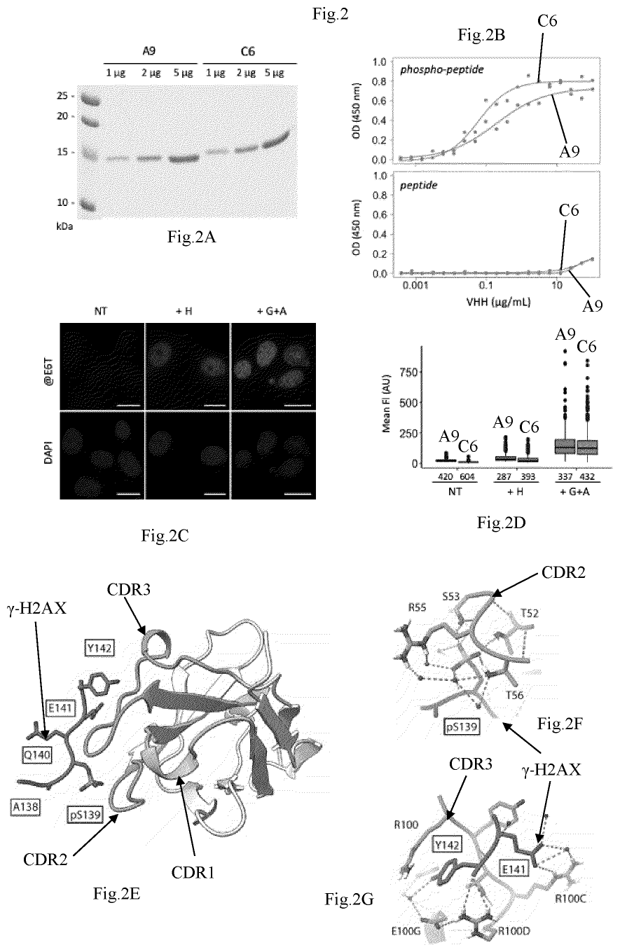

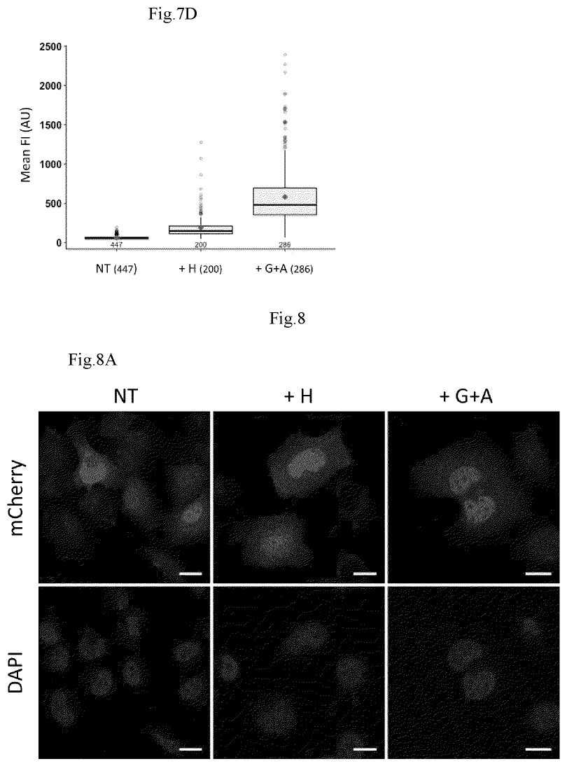

To test whether the four identified VHH variants could be used as nanobodies in immunoassays and cells, the inventors first sub-cloned their coding regions into a bacterial vector equipped with the relevant tags for detection and purification, then expressed them as single polypeptides in the cytoplasm of E. coli cells. SDS-PAGE analysis of cell extracts showed that the four nanobodies behave differently, despite their high amino acid sequence homology: C6 and A9 are soluble, whereas A4 and G2 are mostly insoluble after cytoplasmic expression (FIG. 7A). The yield of the purified A9 and C6 nanobodies, which migrate as single bands on gel (FIG. 2A), was in the range of 8-20 mg/L of bacterial culture. Their capacity to bind to the phospho-peptide was tested by ELISA. Both reacted with the antigen when used at a concentration of 0.1-10 μg/mL (FIG. 2B). No reactivity towards the non-phosphorylated peptide was observed under these conditions (FIG. 2B), confirming the results obtained with the phage-ELISA. The inventors then tested the performance of the purified A9 and C6 nanobodies in immunofluorescence. Both showed the typical staining of γ-H2AX following treatment of H1299 cells with H or with a combination of gemcitabine (G) and a Chk-1 inhibitor (A) (FIG. 2C and FIG. 7B). Treating the cells with both drugs induces intense RS. Interestingly, image quantification indicated that the background staining in this assay was always lowest with C6 (FIG. 2D). Despite their ability to detect the phosphorylated C-terminus of H2AX in fixed cells, these nanobodies could hardly reveal γ-H2AX foci, which were instead readily observed with the well characterized anti-γ-H2AX mAb 3F4 (FIG. 7C) (Moeglin, E. et al.; Cancers 2019, 11, 355, doi:10.3390/cancers11030355). Overall, the results suggested that both C6 and A9 can be solubly expressed at high yields in bacteria and represent hence valuable tools for γ-H2AX detection.

### Example 3: 3D-Structure Determination of the C6 Nanobody

To understand the interaction between the nanobodies and the phospho-peptide at the atomic level, the inventors solved the crystal structure of the complex at 1.8 A resolution. The inventors selected the C6 nanobody due to its higher stability upon storage and overall better performance compared to A9. The crystals belonged to space group P3₁, with 6 equivalent copies of the complex in the asymmetric unit where significant electron density is observed for the last five residues of the peptide (FIG. 2E). The other residues are highly flexible or disordered, implying that they are not involved in specific interactions. The nanobody adopts a canonical IgG fold with a scaffold of nine antiparallel β-strands forming two sandwiching β-sheets. The paratope accepting the phospho-peptide is mainly built from CDR2 and CDR3 resulting in a solvent accessible surface area buried in the interface of approximately 385 Å². Detailed analysis of the complex showed that the phosphate group of phospho-S139 makes direct water-mediated interactions with side chains from CDR2 and CDR3 (FIG. 2F). Key residues (single letter code) that belong to CDR2 are the hydrogen bond donors T52, S53 and T56 as well as R55, which also provides an electrostatic contribution. Interestingly, the nanobody interacts also with the two last residues of the peptide (FIG. 2G). This second binding pocket involves side chains from CDR3 with key roles of R100, R100C and R100D: the ammonium group of R100 is stacked against the aromatic ring of the Y142 tail, while those of R100C and R100D recognize the side chain of E141 and the carboxy-terminal group of the phospho-peptide, respectively. Thus, the phosphate group of the phospho-peptide is a crucial determinant of the recognition of the antigen by the C6 nanobody, explaining its exquisite specificity for the modified peptide.

### Example 4: The C6 and A9 Nanobodies are Solubly Expressed in Mammalian Cells

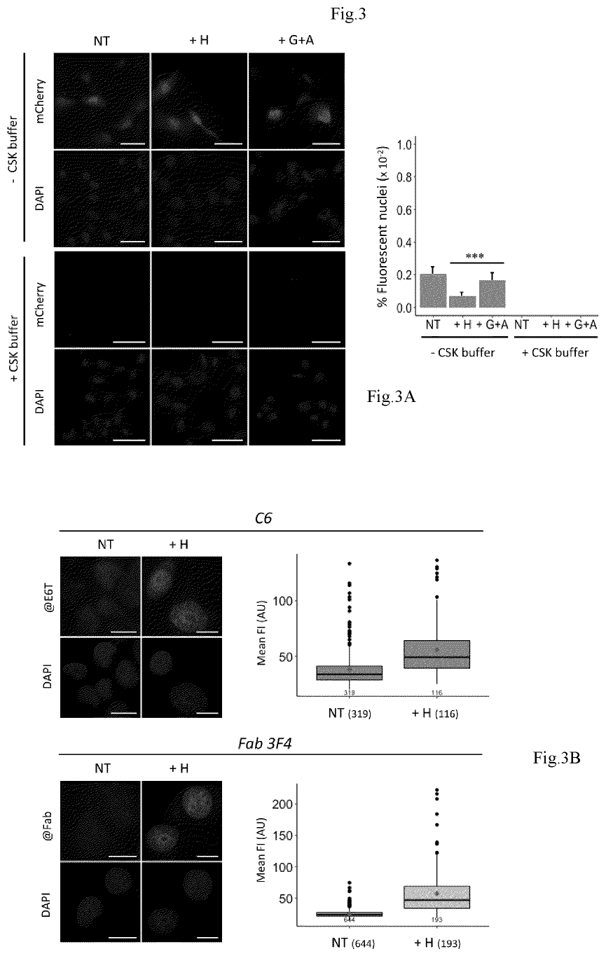

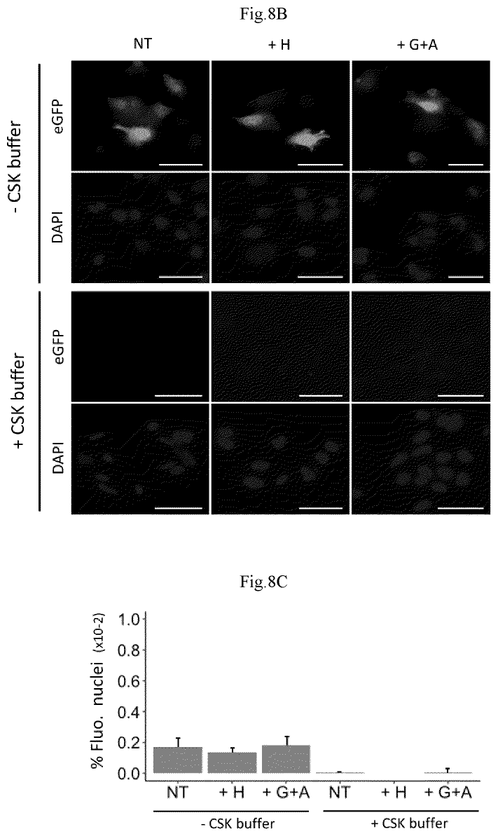

The inventors examined the behavior of the C6 nanobody when expressed in mammalian cells. The inventors cloned the coding region of C6 fused in frame to mCherry to generate a chromobody (Panza, P. et al.; Development 2015, 142, 1879-1884, doi:10.1242/dev.118943) expressed under the control of the R-actin promoter and transiently transfected it into H1299 cells. The C6 chromobody was located in the nucleus of the treated cells as well as the untreated cells after analysis with either a widefield (FIG. 3A) or a confocal microscope (FIG. 8A). The inventors speculated that unspecific binding to a nuclear antigen was caused by the overexpression of the chromobody. To discern specific from unspecific binding, the inventors treated the cells with cytoskeletal (CSK) buffer prior to fixation. This treatment allows washing out all soluble (unbound and/or weakly bound) proteins while retaining the interactions of stably bound material. Under such stringent conditions, the signal of the chromobody was lost in both drug-treated and untreated cells (FIG. 3A), indicating that its nuclear localization upon transfection does not correspond to specific antigen binding and that the affinity of C6 for γ-H2AX may not be sufficient to counteract the CSK wash. The same results were obtained when C6 and mCherry were substituted with A9 and GFP, respectively (FIGS. 8B-8C). Thus, despite their solubility in mammalian cells, these reagents cannot be transfected into cells to detect with precision γ-H2AX after drug treatment likely due to too high chromobody expression levels.

### Example 5: Behavior of the C6 Nanobody Following Transduction

In a previous work the inventors have shown that antibodies and fragments thereof can be efficiently transduced into cultured cells by electroporation (Muyldermans, S.; Annu. Rev. Biochem. 2013, 82, 775-797, doi:10.1146/annurev-biochem-063011-092449; Conic, S. et al.; J. Cell Biol. 2018, 217, 1537-1552, doi:10.1083/jcb.201709153). Given their small size (15-20 kDa), nanobodies can theoretically easily diffuse into the nucleus after delivery in the cytoplasm. Therefore, the inventors transduced the purified C6 nanobody in H1299 cells subsequently treated with H and imaged them after 24 or 48 hours of incubation. As shown in FIG. 3B, the fluorescent signal resembled that typically observed for γ-H2AX, albeit background staining (without treatment) was also significant. Since a similar staining was observed with the transduced Fab prepared by papain digestion of mAb 3F4 (Moeglin, E. et al.; Cancers 2019, 11, 355, doi:10.3390/cancers11030355)(FIG. 3B), the inventors concluded that the C6 nanobody binds to γ-H2AX under physiological incubation conditions. Nevertheless, the background signal of C6 was above that obtained with Fab 3F4 (FIG. 3B, right panels), which may indicate that the recognition of the antigen under these conditions is likely less stable for C6 than for Fab 3F4. Since the inventors' aim was to develop a nanobody that can be used in live cancer cells at low concentrations, the inventors decided to further improve the binding affinity of the C6 nanobody.

### Example 6: The Bivalent C6 Nanobody Allows Highly Accurate Detection of γ-H2AX in Fixed Drug-Treated Cells

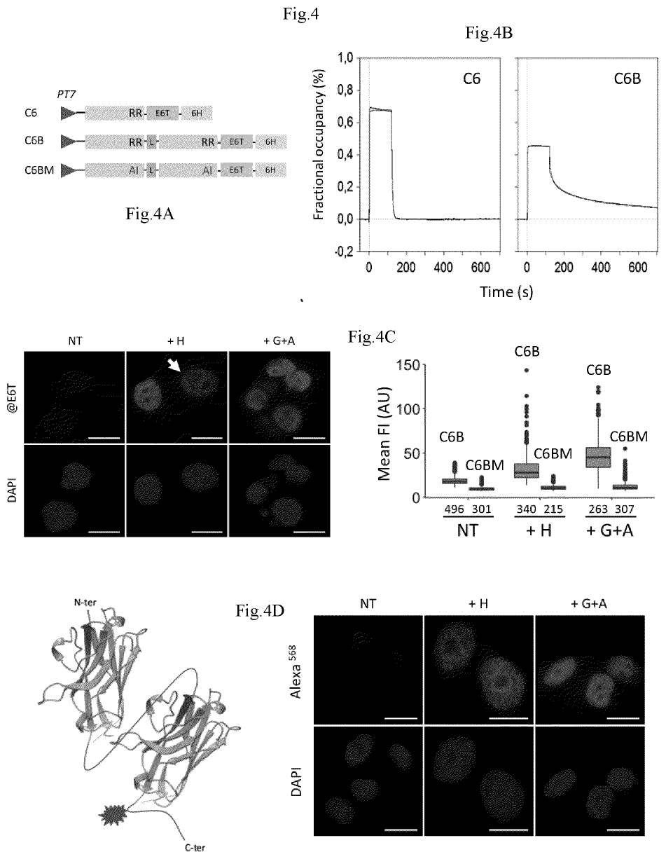

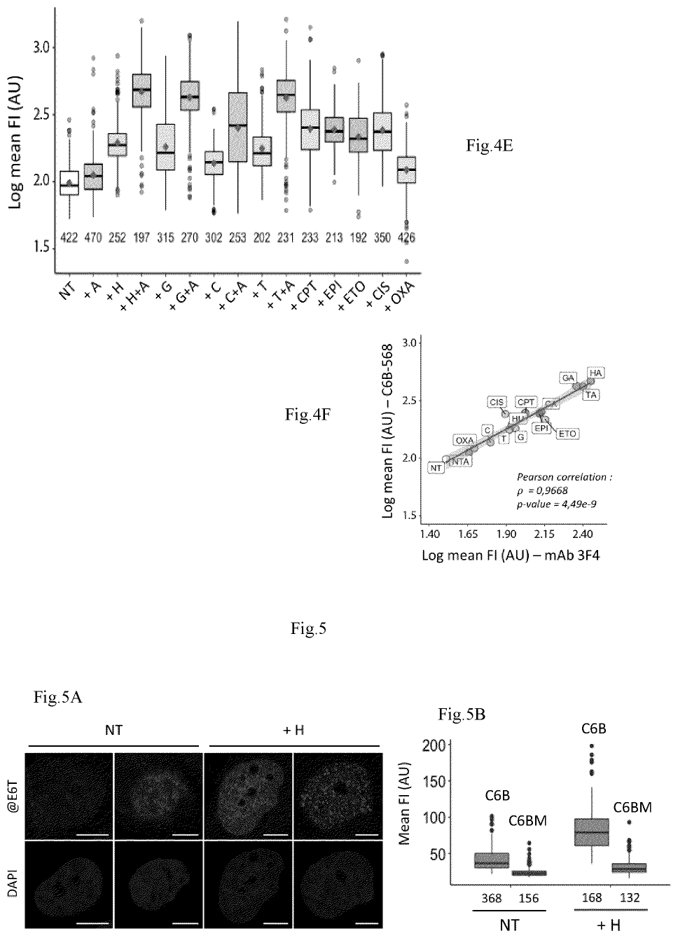

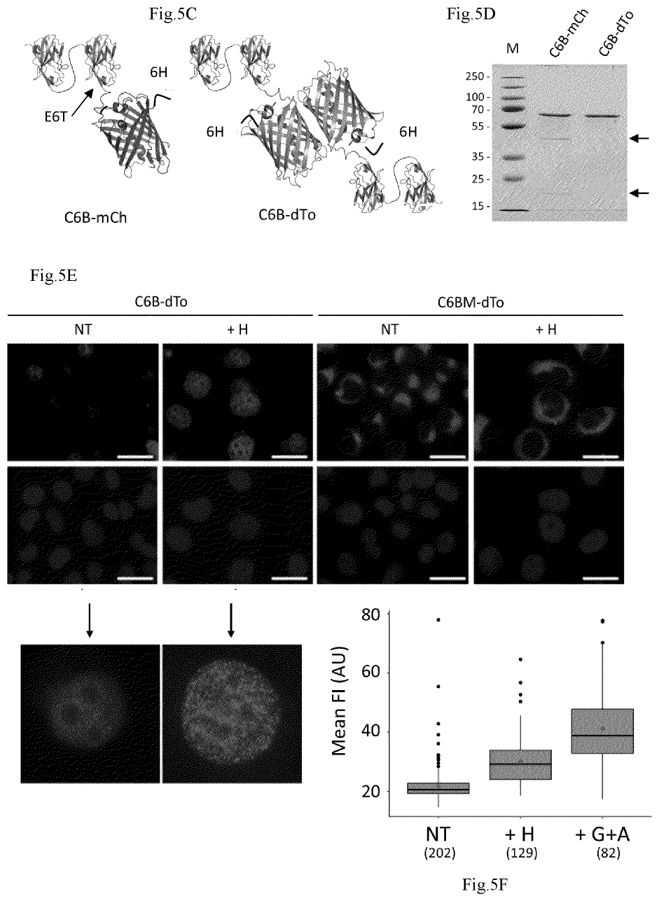

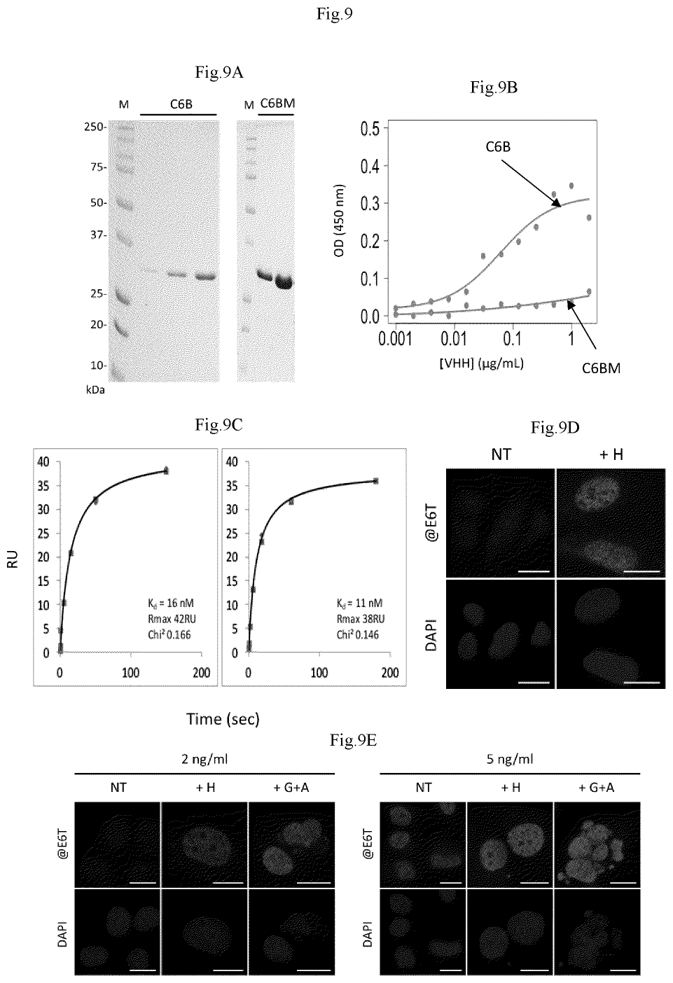

The inventors constructed a bivalent C6 nanobody (called C6B hereafter) and a mutated version of it (called C6BM), where the two R residues at positions 100C and 100D, which are critical for binding (see FIG. 2G), were replaced with alanine and isoleucine, respectively (FIG. 4A). Both constructs were expressed in E. coli cells and, after purification and validation on gel (FIG. 9A), their capacity to bind to the phospho-peptide immobilized on plate was tested by ELISA (FIG. 9B). This experiment showed that C6B bound specifically to the phospho-peptide, whereas C6BM was, as expected, no more reacting.

To assess whether the purified C6B molecules harbor two functional binding sites, the inventors performed quantitative binding assays using the surface plasmon resonance (SPR) technology. To calculate the affinity of C6 for the antigen (K_{D} value) the inventors used purified monovalent molecules and found that it lies in the low nanomolar range (11+/−4 nM; FIG. 9C). To compare the binding properties of the C6 and C6B nanobodies, saturating amounts were injected separately over a surface coated with an equal amount of the phospho-peptide. The responses were normalized to the peptide density and to the nanobody molecular weight, which allows calculating the fractional occupancy (FO) of the peptide sites (Zeder-Lutz, G. et al.; Anal. Biochem. 2012, 421, 417-427, doi:10.1016/j.ab.2011.09.015). At saturation, an FO of one is expected for a 1:1 antibody-antigen molar ratio, while an FO of 0.5 is expected for a homogenous bivalent binding (i.e., 1:2 antibody-antigen molar ratio). As shown in FIG. 4B, the FO of C6B bound to the phospho-peptide was significantly reduced compared to that obtained with C6, indicating that a large proportion of C6B interacts with the antigen in a bivalent manner. These results, in addition to the slower dissociation rate of C6B observed on the sensorgrams (FIG. 4B), strongly suggest that both VHHs comprising C6B are able to bind to the immobilized phospho-peptide.

To examine if this property of binding by avidity represents an advantage for the detection of γ-H2AX in drug-treated H1299 cells, the inventors performed IF experiments as done for the monovalent molecules (FIG. 4C). In this case, upon treatment of the cells with H, γ-H2AX foci could be distinguished more clearly than with the monovalent nanobody (compare FIGS. 2C and 4C). The same result was obtained with H-treated U2OS cells (FIG. 9D), indicating that the bivalent nanobody allows detecting γ-H2AX with high precision in fixed cells. No signal was observed with the C6BM mutant nanobody (FIG. 4C, right panel), which confirms that the staining observed with C6B represents specific binding. Importantly, the staining obtained with C6B when used at a concentration of approximately 2 ng/mL (FIG. 9E) is identical to that observed with mAb 3F4 (FIG. 7C), suggesting that bivalent binding at low concentrations is required to be able to visualize discrete amounts of γ-H2AX in cells.

### Example 7: Single-Step Detection of γ-H2AX in Fixed Drug-Treated H1299 Cells

To test if C6B could be used as a single-step reagent to detect γ-H2AX foci in fixed cells, the inventors added a cysteine residue in the coding region of C6B between the C-terminus of the second VHH and the E6 tag. The purified protein (C6BC) was labelled with Alexa-Fluor 568-maleimide and used in IF (FIG. 4D). γ-H2AX foci could be distinctly detected and quantified with the fluorescently-labelled C6BC molecules when using different combinations of RS-inducing drugs used in the clinic (FIG. 4E). When these results were compared to those obtained with the 3F4 mAb used for the screening of the drugs under similar conditions, a linear correlation was obtained with a Pearson correlation coefficient of 0.966 (FIG. 4F) demonstrating that fluorescently-labelled C6BC performs as well as the validated mAb for detecting γ-H2AX foci in fixed cancer cells.

### Example 8: The Transduced Bivalent C6 Nanobody Allows Monitoring γ-H2AX in Drug-Treated Live Cells

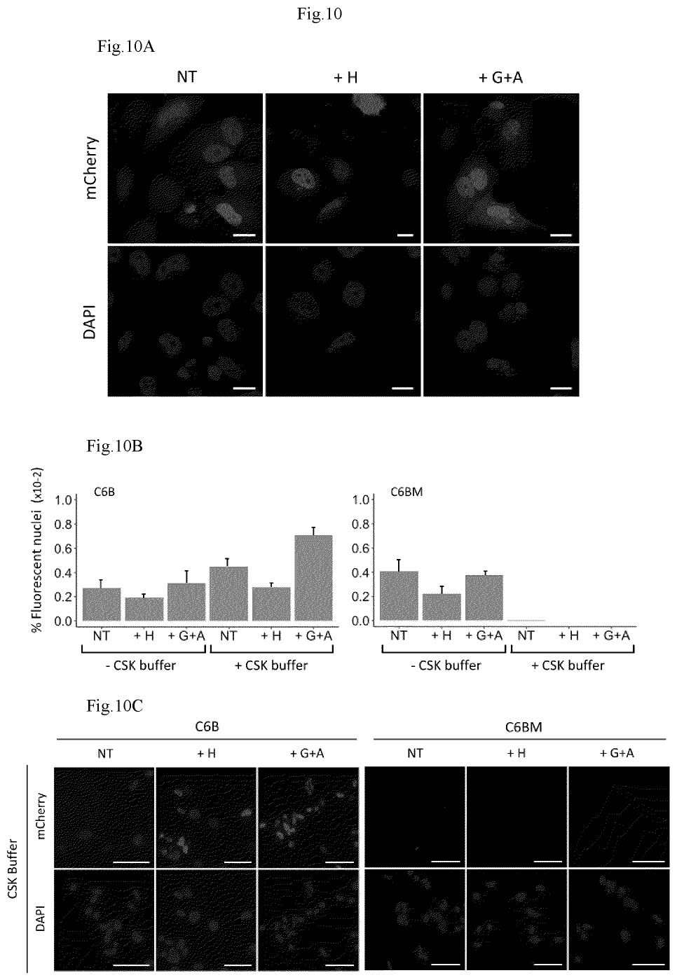

To investigate whether C6B could be used in cells, the inventors modified the previously constructed chromobody C6-mCherry to add a second VHH copy thus creating C6B-mCherry. Upon transfection of H1299 cells with this construct, a strong nuclear mCherry signal was observed (FIG. 10A). In contrast to what observed with IF, nuclear staining was also observed in the absence of drug treatment, indicating a certain degree of unspecific binding. Nonetheless, CSK treatment showed that a large fraction of the fluorescent signal remained in the nucleus (FIG. 10B). No signal was detectable after CSK treatment in cells transfected with the mutant C6BM-mCherry construct (FIG. 10C). These results, together with the fact that the monovalent C6-mCherry molecules were entirely washed off from the nucleus upon CSK treatment (FIG. 3A), clearly indicate that bivalency is of importance for observing binding to the antigen in cells. However, in transient transfection conditions when the plasmid-borne chromobody is highly expressed in cells, unspecific binding remains an issue.

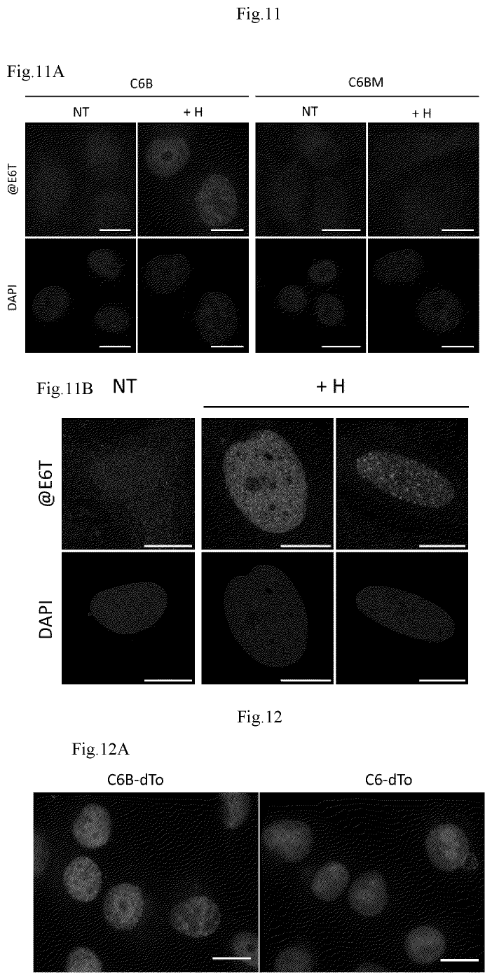

Next, the inventors investigated the performance of the bivalent C6B and C6BM nanobodies by transduction since the corresponding polypeptides showed single bands on gel after purification (FIG. 9A). As shown in FIG. 5A, typical patterns of γ-H2AX in H1299 cells following transduction with C6B were observed by confocal microscopy. In some cells, significant background staining was observed, but that may correspond to the detection of endogenous stress which is relatively high in H1299 cells. In contrast, no staining could be evidenced with C6BM (FIG. 5B and FIG. 11A), indicating that the monitored signal with C6B is specific. Importantly, γ-H2AX could also be specifically detected in H-treated U2OS cells under these conditions (FIG. 11B).

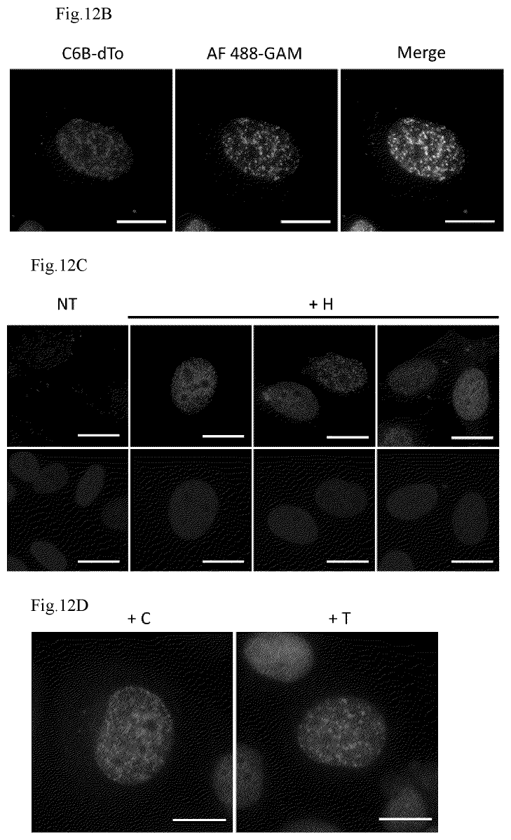

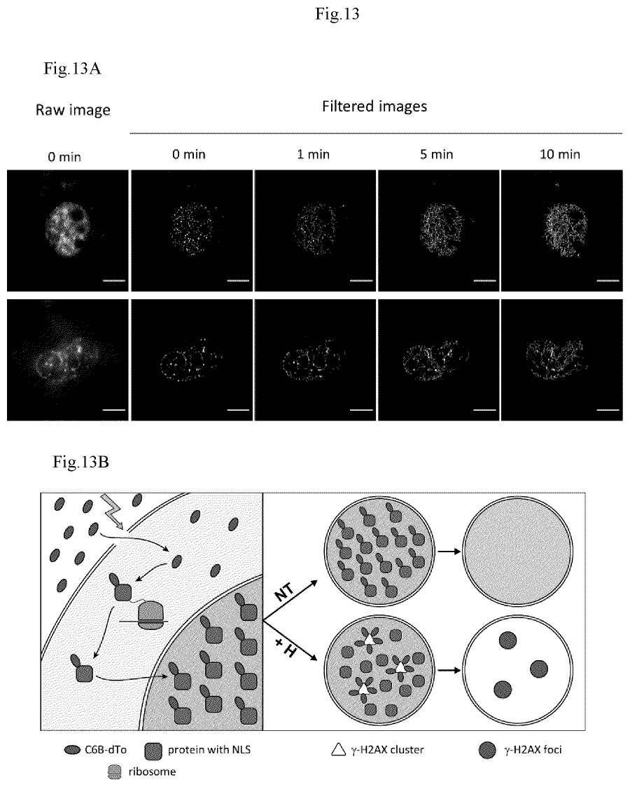

To further analyze whether the inventors could use the C6B chromobody in transduction experiments, the inventors produced C6B-mCherry and C6B-dTomato fusion proteins in E. coli. dTomato protein was tested because its intrinsic fluorescence brightness is approximately 3 times higher than that of mCherry. The expected structures of these molecules are schematically depicted in FIG. 5C. The analysis on gel of these fusion proteins showed that C6B-mCherry was systematically cleaved during the protein preparation, whereas C6B-dTomato protein (referred hereafter to as C6B-dTo) migrated as a single band (FIG. 5D). The delivery in H1299 cells of C6B-dTo led to the specific visualization of foci upon RS induction (FIG. 5E, enlarged micrographs). No nuclear signal was observed with C6BM-dTo and the delivered polypeptides remained in this case in the cell cytoplasm and accumulated next to the nuclear membrane (FIG. 5E). In addition, the inventors observed a more intense fluorescence signal in the nuclei of transduced cells treated with G+A instead of H (FIG. 5F). The inventors also confirmed the importance of bivalency of C6B in this context. Transduction of purified C6-dTo proteins under similar conditions did not allow foci detection and the fusion proteins preferentially accumulated in the nucleoli (FIG. 12A), demonstrating that the specific binding of the bivalent C6 nanobody is maintained within the crowded intracellular context. As a control, the inventors stained the C6B-dTo-transduced cells with mAb 3F4 before microscopic analysis. Notably, the foci detected with C6B-dTo strictly co-localized with those visualized with the antibody and secondary Alexa fluor 488-labelled globulins (FIG. 12B). Since a similar staining pattern was observed in H-treated U2OS (FIG. 12C) and in H1299 cells treated with clofarabine (C) or triapine (T)—two other drugs that target the ribonuclease reductase enzyme as does H—(FIG. 12D), the inventors concluded that the possibility of specific binding through enforced avidity due to the dimerization of the dTomato protein could represent an added value for the true detection of γ-H2AX in the crowded intranuclear space of mammalian cells.

### Example 9: Real-Time Analysis of γ-H2AX in Drug-Treated H1299 Cells

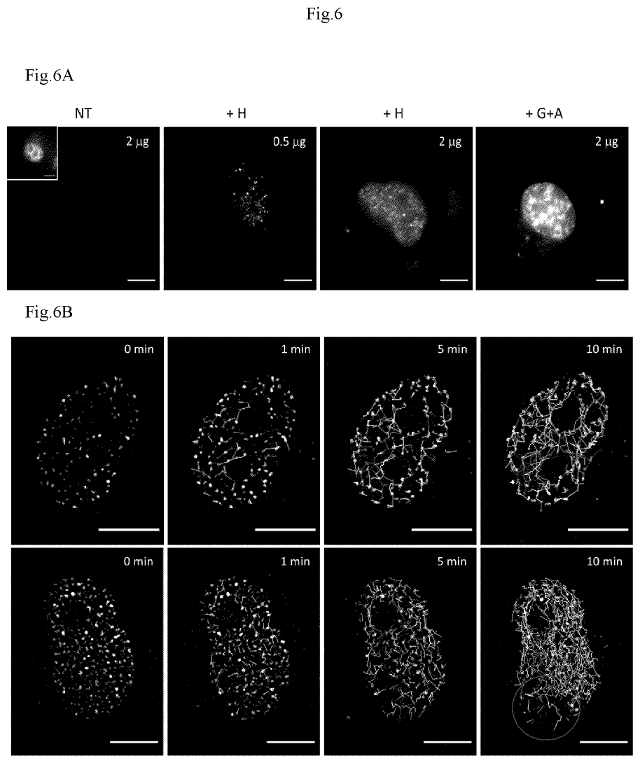

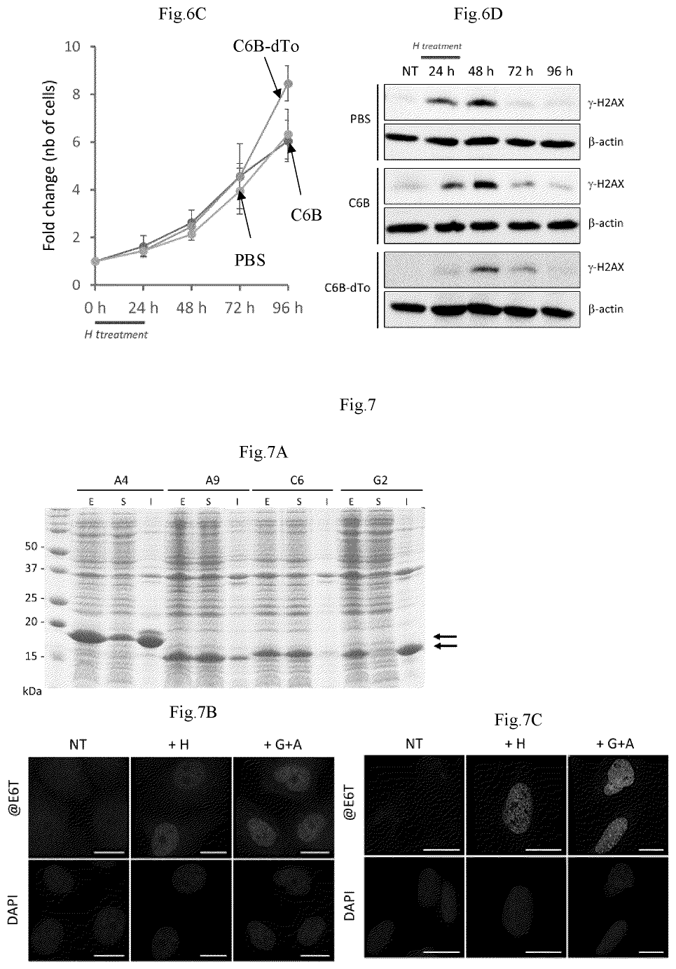

To follow the fate of γ-H2AX foci in live cells, the inventors took advantage of the strong fluorescence signal emitted by the dTomato protein and the fact that precise low amounts of C6B-dTo molecules can be delivered in cells via our electroporation method. Preliminary experiments showed that almost all of the internalized molecules accumulated in the nucleus when 0.5 to 2 μg of purified fusion protein were used. FIG. 6A shows typical nuclei of C6B-dTo-transduced H1299 cells monitored by wide-field microscopy following treatment of the cells with H or G+A for 24 hours. Whereas no foci could be observed in the untreated cells, tiny amounts of them were clearly observed after transduction of 0.5 μg of protein. The signal was higher when 2 μg were used and expectedly even more intense when the cells were treated with G+A instead of H (FIG. 6A). Images were acquired every minute over a period of 10 minutes. Image processing to subtract the background signal (Materials and Methods) allowed us to distinctly record γ-H2AX foci and their movement over time (FIG. 6B). Interestingly, by calculating the trajectories of the foci, the inventors found that those showing bright signal are less mobile that those with low signal. In addition, the foci do not move into the nucleoli and, in some cells, the inventors found that their speed was not homogenous over the whole nucleus (see FIG. 6B, lower panel). The inventors have also checked whether γ-H2AX foci can be observed when lowering the time of incubation of the cells following treatment with G+A (FIG. 13A). Whereas most γ-H2AX-positive nuclei displayed individual foci as observed with H, some of them showed the typical pattern of mid-S phase nuclei (FIG. 13A, lower panel) that has been observed after transfection of cells with a PCNA-GFP construct (Leonhardt, H. et al.; J. Cell Biol. 2000, 149, 271-280, doi:10.1083/jcb.149.2.271). Collectively, the data show that γ-H2AX foci can be imaged without ambiguity in live cells after transduction of C6B-dTo, enabling to study the dynamics of this particular histone modification.

### Example 10: Impact of the Delivered C6B Nanobody on Cell Survival

To assess if the delivered C6B nanobody interferes with the cell response to genotoxic drugs, the inventors performed cell survival assays with transduced H1299 cells and monitored the γ-H2AX levels following pulse-treatment with H for 24 hours. Cells transduced with either PBS, C6B or C6B-dTo grew similarly at day 1, 2 and 3 post-treatment with H (FIG. 6C). This suggests that the delivered C6B molecules are not toxic. Furthermore, Western blotting showed that phosphorylation of H2AX was maximal after 24 hours post pulse-treatment and almost undetectable after 2 days of drug withdrawal (FIG. 6D). This correlates well with the regrowth of the transduced cells and indicates that the binding of the C6B nanobody does not interfere with the cell response to H. The fact that C6B is somewhat non-neutralizing in the cells as it does not interfere with the γ-H2AX turnover makes it an extremely powerful tool for imaging.

### MATERIALS AND METHODS

### VHH Libraries and Phage Selection

Alpacas (Llama pacos) were immunized at days 0, 21 and 35 with the synthetic phosphorylated peptide CKATQA(p)SQEY corresponding to the C-terminus of H2AX after covalent cross-linking to ovalbumin (150 μg). The immunogen was mixed with Freund complete adjuvant for the first immunization and with Freund incomplete adjuvant for the following immunizations. The immune response was monitored by titration of serum samples by ELISA with immunizing peptide on plate. Bound antibodies were detected with anti-alpaca rabbit IgG (Lafaye, P. et al.; Mol. Immunol. 2009, 46, 695-704, doi:10.1016/j.molimm.2008.09.008). For the construction of the libraries, blood samples (200 ml) of the immunized animals were collected under strict veterinary control and the PBMCs were isolated by Ficoll gradient centrifugation (GE Healthcare, Vélizy-Villacoublay, France). For the preparation of total RNA, approximately 10⁷ cells were lysed with the TRlzol reagent (ThermoFisher Scientific, Grand Island, NY, USA). Complementary DNA (cDNA) was amplified using either SuperScript IV reverse transcriptase (ThermoFischer Scientific) or the BD Smart RACE kit (BD Biosciences). The VHH repertoires were amplified from the cDNA by two successive PCR reactions using 3 different primer pairs (PCR1, PCR2; Table D) and the VHH fragments were cloned into the SfiI/NotI restriction sites of the pHEN1 phagemid vector. After transformation into either E. coli TG1 or XL1-blue cells by electroporation, the bacterial colonies (approximately 4×10⁷ independent transformants per library) were infected with M13KO7 helper phage to produce the phage libraries. The recombinant phages of each library were purified by PEG 8,000/NaCl precipitation and aliquots were stored at −80° C. after addition of 15% glycerol. Biopanning was performed with the phospho-peptide (0.5-5 μg/ml) coated on microtiter wells (ThermoFisher Scientific). Briefly, approximately 10¹¹ phages in PBS containing 5% nonfat-dried milk were added to uncoated wells for 1 h and subsequently transferred to the peptide-coated wells. After incubation at 20° C. for 1 hour, the wells were extensively washed with PBS containing 0.05% Tween 20. Bound phages were eluted with trypsin and amplified in growing TG1 cells for the next round of selection. The amount of phospho-peptide coated on plate was lowered to 0.5 μg/ml in the second round of selection. Phage titers and enrichment after each panning round were determined by infecting TG1 cells with 10-fold serial dilutions of the collected phages and plating on LB agar plates containing 100 μg/mL ampicillin and 1% glucose. Where indicated, binding of the phages to antigen on plate was revealed with an anti-M13 monoclonal antibody conjugated to horse radish peroxidase (HRP; Abcam, Cambridge, UK). The VHH nucleotide sequences were determined using the M13-RP primer (GATC-Eurofins, Ebersberg, Germany).

TABLE D
List of the primers used for the construction of the VHH libraries and for generating the pET- and
pβ-actin-based expression vectors

| Name | Nucleotide sequence Used | for generating |
|---|---|---|
| VHBACK-A6 | 5′-GATGTGCAGCTGCAGGCGTCTGGRGGAGG-3′ | VHH-PCR1 |
| CH2FORTA4 | 5′-CGCCATCAAGGTACCAGTTGA-3′ | VHH-PCR1 |
| CALL001 | 5′-GTCCTGGCTGCTCTCTACAAGG-3′ | VHH-PCR1 (ref) |
| CALL002 | 5′-GGTACGTGCTGTTGAACTGTTCC-3′ | VHH-PCR1 (ref) |
| AlpVh-L | 5′-CTGAGCTTGGTGGTCCTGGCTGC-3′ | VHH-PCR1 (ref) |
| Bq-lead-1g-F | 5′-GTCCTGGCTGCTCTWYTACARGG-3′ | VHH-PCR1 |
| Bq-CH2-ca2-R | 5′-GGTACGTGCTGTTGAACTGTTCC-3′ | VHH-PCR1 |
| SM017 | 5′-CCAGCCGGCCATGGCTCAGGTGCAGCTGGTGGAGTCTGG-3′ | VHH-PCR1 |
| SM018 | 5′-CCAGCCGGCCATGGCTCAGGTGCAGCTGGTGGAGTCTGG-3′ | VHH-PCR1 |
| VHBACKA4 | 5′-CATGCCATGACTCGGGGCCCAGCCGGCCATGGCGAKGTSCAGCT-3′ | VHH-PCR2 |
| VHFOR36 | 5′-CATGCCATGACTCGGGGCCCAGCCGGCCATGGCGAKGTSCAGCT-3′ | VHH-PCR2 |
| Bq-FR1-long-F | 5′-GTCATTGGCCCAGCCGGCCATGGCTCAGKTGCAGCTCGTGGAGTCNGG-3′ | VHH-PCR2 |
| Bq-Hin-short-F | 5′-GACATTGCGGCCGCGCTGGGGTCTTCGCTGTGGTG-3′ | VHH-PCR2 |
| Bq-Hin-long-R | 5′-GACATTGCGGCCGCTGGTTGTGGTTTTGGTGTCTTGGG-3′ | VHH-PCR2 |
| E6T-For | 5′-CTAGTATGTTTCAGGATCCAGAACGTCCGCGCG-3′ | pETOM |
| E6T-Back | 5′-CTAGCGCGCGGACGTTCCTGCGGATCCTGAAACATA-3′ | pETOM |
| VHH-BspH1-For | 5′-AACGAACTCATGACTCAGKTGCAGCTCGTGGAGTCGGG-3′ | pET-C6B |
| VHH-BspH1-Deg | 5′-AACGAACTCATGACYSABBTSCAGCTSSWGSMGTCVCC-3′ | pET-C6B |
| VHH-Not1-short | 5′-GGACTAGTTGCGGCCGCTGAGGAGACGGTGACCTG-3′ | pET-C6B |
| VHH-Not1-long | 5′-GGACTAGTTGCGGCCGCTGGTTGTGGTTTTGGTGTTTCGGG-3′ | pET-C6B |
| pETOM-For | 5′-GGAGACCACAACGGTTTCCC-3′ | pETOM |
| pET-Rev | 5′-TTCGGGCTTTGTTAGCAGCC-3′ | pETOM |
| b-actin-For | 5′-GGCTCACAGCGCGCCCGGCT-3′ | pET-C6B |
| G4S-Rev | 5′-CGATCCGCCACCGCCGCTGCCACCTCCGCCTGAACCGCCTCCACCGGCCGC | pET-C6B |
| | TGAGGAGACGGTGA-3′ | |
| G4S-For | 5′-GGTGGAGGCGGTTCAGGCGGAGGTGGCAGCGGCGGTGGCGGATCGATGAC | pET-C6B |
| | TGAGGTGCAGCTGGT-3′ | |
| E6Tag-Rev | 5′-TCCTGCGGATCCTGAAACAT-3′ | pET-C6B |
| C6-Cys-Rev | 5′-AAAAAATGCGGCCGCACATGAGGAGACGG-3′ | pET-C6B |
| C6-Mut-For | 5′-GCAGATATACCGCGATACAGTCTGAG-3′ | pET-C6 |
| C6-Mut-Rev | 5′-CTCAGACTGTATCGCGGTATATCTGC-3′ | pET-C6 |
| mCher-Rev | 5′-CTTGTACAGCTCGTCCATGCC-3′ | pET-C6 |
| mCher-pET-For | 5′-ATTTATGCTAGCGGAGGGATGGTGAGCAAGGGC-3′ | pET-C6B-mCherry |
| mCher-pET-Rev | 5′-ATTTATGCTAGCACTACCCTTGTACAGCTCGTCC-3′ | pET-C6B-mCherry |
| dTo-Bam-For | 5′-GCGCATGGATCCTATGGTGAGCAAGGGCGAGGAG-3′ | pET-C6B-dTo |
| dTo-Bam-Rev | 5′-GCGCGCGGATCCCCGGTGCTGCCGGTGCCATG-3′ | pET-C6B-dTo |
| E6T-Hind-For | 5′-CTAGTATGTTTCAGGATCCGCAGGAACGTCCGCGCAAGCTTG-3′ | pbA-VHH-ET |
| E6T-Hind-Rev | 5′-CTAGCAAGCTTGCGCGGACGTTCCTGCGGATCCTGAAACATA-3′ | pbA-VHH-ET |
| mCherry-For | 5′-ATTTATAAGCTTAGTGGGATGGTGAGCAAGGGC-3′ | pbA-VHH-ET-mC |
| mCherry-Rev | 5′-ATTTATGAATTCTCATTACTTGTACAGCTCGTCC-3′ | pbA-VHH-ET-mC |
| NESPKIa-Hind-For | 5′-AGCTTAACGAGCTCGCTCTCAAACTCGCTGGACTCGACATCAACAAGACCA-3′ | pbA-C6B |
| NESPKIa-Hind-Rev | 5′-AGCTTGGTCTTGTTGATGTCGAGTCCAGCGAGTTTGAGAGCGAGCTCGTTA-3′ | pbA-C6B |

Respectively, SEQ ID Nos: 39-78

### VHH Engineering and Bacterial Production

The coding region of the selected VHHs in the pHEN1 vector were amplified by PCR with primers VHH-BspHI-Deg and VHH-NotI-short and subcloned into the pET-E6T-6H expression plasmid, a derivative of pETOM (Desplancq, D. et al.; J. Immunol. Methods 2011, 369, 42-50, doi:10.1016/j.jim.2011.04.001), which contains in frame at the NotI site the E6 epitope tag recognized by the 4C6 mAb and a His6 tag. To generate the bivalent VHH constructs, the coding region of the VHH was amplified by SOE-PCR with the primer pairs pETOM-For/G4S-Rev and G4S-For/E6T-Rev. The G4S-Rev and G4S-For are the annealing primers to add the (G4S)₃ linker region. The recombinant fragment was cloned into the NcoI-digested pET-C6-E6T-6H plasmid after digestion with NcoI restriction enzyme, thus generating pET-C6B-E6T-6H. To generate the C6 mutant construct, which harbors an Ala residue and an Ile residue instead of the two Arg residues at position 100C and 100D in the CDR3 region, the inventors amplified by SOE-PCR the coding region of the C6 with primers pETOM-For and pETOM-Rev, in combination with C6-Mut-Rev and C6-Mut-For as annealing primers. The resulting PCR fragment was sub-cloned into the NcoI/NotI-digested pET-C6-E6T-6H to obtain pET-C6M-E6T-6H. The plasmid pET-C6BM-E6T-6H, which encodes the bivalent form of the mutated C6 coding region was constructed as described above. The additional Cys residue in the coding region of the bivalent C6 was obtained by amplification of the C6 coding region with primers VHH-BspHI-For and C6-Cys-Rev and sub-cloned into the pET-C6B-E6T-6H.

The pET-C6B-mCherry and pET-C6B-dTomato plasmids were constructed by inserting in frame the coding regions of mCherry protein or dTomato protein in the unique BamHI located in the E6 tag region. The dTomato coding region was subcloned from the ptdTomato-N1 vector (Clontech, Mountain View, USA). All primers used to generate the above-described plasmids are listed in Table D.

The VHH variants were expressed in E. coli BL21(DE3) plysS cells by addition of IPTG (1 mM) and incubation overnight at 20° C. The expressed polypeptides were purified as previously described (Desplancq, D. et al.; J. Immunol. Methods 2011, 369, 42-50, doi:10.1016/j.jim.2011.04.001), except that IMAC chromatography was performed on a HITRAP™ IMAC HP 1 ml column (GE Healthcare) loaded with nickel ions. The C6 variants with a cysteine residue at the C-terminal end of the second VHH coding region were purified on HIS TRAP™ Excel columns (GE Healthcare) in the presence of 2 mM TCEP. All buffers used in the process were supplemented with 1 mM EDTA and 0.2 mM PMSF. Where indicated, the eluted samples were further purified by size exclusion chromatography on a Superdex 75 10/300 GL column equilibrated in 20 mM Hepes buffer pH 7.2 containing 50 mM NaCl, 1 mM EDTA, 0.1 mM PMSF and 2 mM TCEP (optional). The C6B-mCherry and C6B-dTomato fusion proteins were purified by IMAC chromatography on HITRAP™ columns as above and subsequently polished by size exclusion chromatography on a HILOAD 16/600 Superdex 200 PG column (GE Healthcare) equilibrated in PBS. All purified proteins were stored at −80° C. after addition of 10% glycerol.

### ELISA

For the ELISA assays, microtiter wells (ThermoFisher Scientific) were coated with 1 μg/mL of phosphorylated or non-phosphorylated peptide CKATQASQEY in PBS overnight at 4° C. The purified VHH preparations were diluted in PBS containing 0.2% non-fat died milk and following incubation at RT for 1 hour they were revealed with mAb 4C6 and subsequent addition of HRP-conjugated rabbit anti-mouse IgG (GE Healthcare). After several washes with PBS containing 0.1% NP40 and addition of 3′,3′,5′,5′-tetramethylbenzidine (Sigma-Aldrich), the optical density was measured at 450 nm in an ELISA reader. The data were processed with R software using the drc package (Ritz, C. et al.; PLoS ONE 2015, 10, e0146021, doi:10.1371/journal.pone.0146021).

### SPR Analysis

All experiments were performed on a Biacore T200 instrument at 25° C. in HBS-P buffer containing 10 mM HEPES (pH 7.4), 150 mM NaCl, 0.05% P20 surfactant. The phospho-peptide CKATQA(p)SQEY was immobilized on the biosensor surface (BR-1005-30; GE healthcare) through the SH group of the N-terminal cysteine using thiol coupling chemistry. The reference surface was treated similarly except that peptide injection was omitted. The purified VHH samples were serially injected in duplicate for 120 seconds over reference and peptide surfaces. Each sample injection was followed by a wash with HBS-P buffer during 600 sec. Sensorgrams were corrected for signals from the reference flow cell as well as after running buffer injections. The Kd was determined by fitting the equilibrium response (Req) versus the concentration curve to a 1:1 interaction model with the Biacore 2.0.2 evaluation software (GE Healthcare). Responses were normalized relative to phospho-peptide density as fractional occupancy (FO) of target sites (Zeder-Lutz, G. et al.; Anal. Biochem. 2012, 421, 417-427, doi:10.1016/j.ab.2011.09.015).

### 3D Structure Determination

Purified C6 protein was incubated for 1 hour with a 1.3-fold excess of phospho-peptide treated with 2 mM N-ethyl maleimide to prevent dimerization. The complexes were subjected to size exclusion chromatography on a Superdex 75 10/300 GL column (GE Healthcare) equilibrated in 20 mM Hepes buffer pH 7.2, 150 mM NaCl. The peak fractions were concentrated to 5.1 mg/ml with a Amicon Ultra 3K filter (Merck-Millipore). The crystallization experiments were carried out by the sitting-drop vapor diffusion method at 20° C. using a Mosquito Crystal dispensing robot (TTP Labtech) for mixing equal volumes (200 nL) of the C6-peptide sample and reservoir solutions in 96-well 2-drop MRC crystallization plates (Molecular Dimensions). Crystallization conditions were tested using commercially available screens (Qiagen, Molecular Dimensions). Several wells were found positive after about 1 week of incubation and crystals obtained with 25% PEG 3350, 0.2M sodium acetate. The crystals were transferred to 35% PEG 3350, 0.2M sodium acetate before being flash cooled in liquid nitrogen. The data were collected at the Proxima 2A beamline of the synchrotron Soleil at a wavelength 0.98 Å (12.65 keV) on an EIGER X 9M detector (Dectris) with 20% transmission. 360° of data were collected using 0.1° oscillation and 0.025 s exposure per image, with a crystal to detector distance of 134.25 mm. The data were indexed, integrated, and scaled using XDS (Kabsch, W.; Acta Crystallogr. D Biol. Crystallogr. 2010, 66, 125-132, doi:10.1107/S0907444909047337). The 3D structure of the C6/phosphopeptide complex was solved by molecular replacement using the PHASER module of PHENIX (Liebschner, D. et al.; Acta Crystallogr. D Struct. Biol. 2019, 75, 861-877, doi:10.1107/S2059798319011471) with the structure of VHH PorM_01 (PDB ID: 5LZO) edited to remove water molecules and the CDR loops, being used as a search model. Refinement was performed using the refine module of PHENIX followed by iterative model building in COOT (Emsley, P. et al.; Acta Crystallogr. D Biol. Crystallogr. 2010, 66, 486-501, doi:10.1107/S0907444910007493). The structural figures were prepared with Chimera-X software (http://www.rbvi.ucsf.edu/chimerax).

### Cell Culture, Transduction, Histone Preparation and Western Blotting

The H1299 and U2OS cells (laboratory stocks) were maintained in Dulbecco's modified Eagle's tissue culture medium (DMEM; Life Technologies, Carlsbad, USA) supplemented with L-glutamine (2 mM), gentamicin (50 μg/mL) and 10% heat inactivated fetal calf serum at 37° C. in a humidified 5% CO₂ atmosphere. Fresh cells were thawed from frozen stocks after 10 passages and mycoplasma contamination was tested by DAPI staining. Counting of the cells was performed with the automated cell counter LUNA-II (Logos Biosystems, Villeneuve d'Ascq, France). Where indicated, the cells were treated with either hydroxyurea (H; 2 mM), gemcitabine (G; 0.1 μM), AZD-7762 (A; 0.1 μM), clofarabine (C; 0.3 μM), triapine (T; 2 μM), camptothecin (CPT, 1 μM), epirubicin (EPI, 0.5 μM), etoposide (ETO, 10 μM), cisplatin (CIS, 10 μM), oxaliplatin (OXA, 10 μM) or combinations of two drugs at the same concentration as indicated. All drugs were purchased from Sigma-Aldrich.

Transduction experiments with purified C6, C6B, C6B-dTomato proteins or Fab 3F4 were performed essentially as previously described (Freund, G. et al.; mAbs 2013, 5, 518-522, doi:10.4161/mabs.25084). Briefly, 10⁵ cells in PBS were mixed with the protein sample (0.5-2 μg) and subjected to electroporation (1550 V, 10 msec, 3 pulses) using the Neon transfection device (Life Technologies). The treated cells were incubated for 1 hour at 37° C. in medium and, after centrifugation for 5 min at 100 g, the pelleted cells were seeded and allowed to recover overnight in complete medium without antibiotics before addition of the drugs.

For the purification of the histone proteins, the harvested cells (approximately 10⁷/ml) were lysed for 10 minutes at 4° C. in PBS supplemented with 0.5% Triton X100, 2 mM PMSF, 0.02% NaN₃ and 1 mM Na₃VO₄. After centrifugation for 10 minutes at 6500 g at 4° C., the recovered nuclei were acid extracted overnight at 4° C. in 0.2 M HCl. The histone proteins present in the clarified lysate were stored at −20° C.

For the analysis of the H1299 proteins by Western blotting, soluble extracts (60 μg/lane) in RIPA buffer were used. γ-H2AX and β-actin were revealed with monoclonal antibody 3F4 (0.1 μg/mL) and rabbit polyclonal serum A2066 (Sigma-Aldrich), respectively. Bound secondary HRP-labeled antibodies were revealed with ECL reagent (GE Healthcare) and analyzed with the Image QuantLAS 4000 imager (GE Healthcare).

Construction of the pβ3-actin plasmids and transient transfection

The pβA-scFv-eGFP, a derivative of pDRIVE-hβ-actin (Rinaldi, A.-S. et al.; Exp. Cell Res. 2013, 319, 838-849, doi:10.1016/j.yexcr.2013.01.011) was modified by PCR to insert in frame to the scFv the E6 tag and the mCherry protein using E6T-HindIII-For/E6T-HindIII-Rev and mCherry-For/mCherry-Rev primer pairs, respectively. This vector which carries unique NcoI and SpeI restriction sites was used to sub-clone the VHH variants as described above, thereby generating pβA-C6-E6T-mCherry, pβA-C6M-E6T-mCherry, pβA-C6B-E6T-mCherry and pβA-C6BM-E6T-mCherry. All oligonucleotides used to construct these expression vectors are listed in Table D.

The day before transfection, 8×10⁴ cells were plated in 12-well culture plates containing glass coverslips. Transient DNA transfection was performed using jetPRIME (Polyplus Transfection, Illkirch, France) according to manufacturer's instructions. The culture medium was replaced with fresh medium after 4-24 hours of incubation with the polymer/plasmid mixtures. Cells were incubated (37° C., 5% CO2) for 40 hours (H-treated cells) or 24 hours (G+A-treated cells), followed by microscopic analysis.

### Immunofluorescence Microscopies

For the analysis by classical immunofluorescence microscopy, the transfected or transduced cells were fixed with 4% paraformaldehyde for 20 minutes and, after permeabilization with 0.2% Triton X100 for 5 min, they were incubated with mAb 3F4 or VHH preparations diluted in PBS containing either 10% fetal calf serum or 2% BSA. Where indicated, the cells were treated with CSK-100 modified buffer (100 mM NaCl, 300 mM sucrose, 3 mM MgCl₂, 10 mM HEPES pH 6.8, 1 mM EGTA, and 0.2% Triton X-100) for 5 minutes prior to fixation. The VHH molecules were revealed by addition of mAb 4C6 which binds to the E6 tag and bound antibodies were detected with Alexa Fluor 488 or 568 labelled-anti-mouse immunoglobulins (Life Technologies). Where indicated, Alexa 568 labelled-C6B molecules were used. The labelling of the purified bivalent C6 equipped with a cysteine residue at the C-terminus was performed essentially as previously described (Shaner, N.C. et al.; Nat. Biotechnol. 2004, 22, 1567-1572). Briefly, purified protein in 0.1 M KH₂PO₄ pH 6.5, 150 mM NaCl, 1 mM EDTA, 250 mM sucrose was mixed with a 1.2 molar amount of Alexa Fluor 568 maleimide derivative (ThermoFisher Scientific). After adjustement of the pH at 7.5 and subsequent incubation for 1 hour at room temperature, the chemical reaction was blocked with N-ethylmaleimide in excess. The mixture was centrifuged through either a Zeba spin column with a cut-off of 7 kDa (GE Healthcare) or the fluorescent dye removal column provided by Thermofischer Scientific. The amount of fluorophore per bivalent C6 in the flow-through was calculated by spectrophotometry with a Nanodrop 2000 device (ThermoFisher Scientific). After incubation of the cells with the different reagents and several washes with PBS, the coverslips were mounted with 4′,6′-diamino-2phenyl-indole (DAPI) Fluoromount-G (Southern Biotech, Birmingham, USA) and imaged with a Leica DM5500 microscope (Leica, Wetzlar, Germany) equipped with 20× and 63× objectives. The signal was recorded with a Leica DFC350FX camera. Confocal microscopy was performed as previously described (Conic, S. et al.; J. Cell Biol. 2018, 217, 1537-1552). All microscopy images were processed using the Fiji/Image J software. For the measurement of the nuclear fluorescence intensity, the images of microscopy fields were acquired with the 20× objective. The nuclei were set with the DAPI channel acquisition as regions of interest (ROI) and the mean fluorescence intensity in each ROI was measured using the Fiji built-in-tool and data were processed with the R software.

Widefield fluorescence microscopy was performed on a home-built system composed by a Nikon TiE inverted microscope coupled to a high-numerical aperture (NA) TIRF objective (Apo TIRF 100X, oil, NA 1.49, Nikon). Live-sample were illuminated with a laser diode at 561 nm (10 W/cm², Oxxius) at 37° C. Real-time imaging was performed by introducing a single edge dichroic mirror and a bandpass filter in the emission path of the microscope (Semrock, 560 nm edge BrightLine single-edge imaging-flat dichroic beamsplitter, 593/40 nm BrightLine single-band bandpass filter) and by using an EM-CCD camera (ImagEM, Hamamatsu, 0.106 μm pixel size) with a typical integration time of 100 ms. The videos were recorded using the perfect focus system of the microscope to avoid z-drift during the acquisition (1 image recorded every minute during 10 minutes). Images were processed using Fiji. To visualize the movement of the foci, the inventors used a filtering procedure in which two different Gaussian blurs (A=1.3 pixel and B=2 pixels) were applied to each image of the stack. The improved stack was obtained by computing the difference between A and B. The Mosaic plugin was then used on the final stack to reconstruct the single foci trajectories over the whole acquisition.

### Statistical Analysis

Statistical analysis was performed using R software version 3.6.1. Averages are represented as means+/−SD and the number of replicates is indicated in the figure legends. In the boxplots (FIGS. 2-5), the bars indicate the median and interquartile range of the recorded fluorescence after processing with R software. The statistical significance of the data obtained after transfection (FIG. 3) was determined with the Student's t test and indicated as ***p-value <0.001. Prior to the Student's t test, normality and equality of variances were tested using Shapiro-Wilk's test and Fisher's F-test respectively. For the correlation analysis (FIG. 4), normality of the data was tested using Shapiro-Wilk's test and correlation was evidenced by calculating the Pearson's correlation coefficient.

1-24. (canceled)

25. A single domain antibody directed against H2AX with a phosphorylation of serine at position 139 (γ-H2AX) comprising a variable domain comprising three complementarity determining regions (CDRs), namely CDR1, CDR2 and CDR3, consisting in the amino acid sequence of SEQ ID NO: 1: GLT(L/F)SRYA for CDR1, the amino acid sequence of SEQ ID NO: 2: ITASGRTT for CDR2, and the amino acid sequence of SEQ ID NO: 3: AADYGX₁X₂X₃YTRRQSEYX₄Y for CDR3, wherein X₁ and X₂ are any amino acid, X₃ is K or R, and X₄ is D or E.

26. The single domain antibody according to claim 25, wherein X₁ and X₂ are independently selected from the group consisting of A, V, S, N, K, R, T and G.

27. The single domain antibody according to claim 25, wherein X₁ is selected from the group consisting of S, N, T and G and/or X₂ is selected from the group consisting of G, K and S.

28. The single domain antibody according to claim 25, wherein X₁ is selected from the group consisting of S, N, T and G; X₂ is selected from the group consisting of G, K and S; X₃ is R; and X₄ is E.

29. The single domain antibody according to claim 25, wherein the amino acid sequence of CDR3 is selected from the group consisting of:
(SEQ ID NO: 4)
AADYGSGKYTRRQSEYDY;
(SEQ ID NO: 5)
AADYGNKRYTRRQSEYEY;
(SEQ ID NO: 6)
AADYGTSRYTRRQSEYEY;
(SEQ ID NO: 7)
AADYGGGRYTRRQSEYEY;
(SEQ ID NO: 8)
AADYGSGRYTRRQSEYDY;
(SEQ ID NO: 9)
AADYGSGKYTRRQSEYEY;
(SEQ ID NO: 10)
AADYGSGRYTRRQSEYEY;
(SEQ ID NO: 11)
AADYGNKKYTRRQSEYEY;
(SEQ ID NO: 12)
AADYGNKRYTRRQSEYDY;
(SEQ ID NO: 13)
AADYGNKKYTRRQSEYDY;
(SEQ ID NO: 14)
AADYGTSKYTRRQSEYEY;
(SEQ ID NO: 15)
AADYGTSRYTRRQSEYDY;
(SEQ ID NO: 16)
AADYGTSKYTRRQSEYDY;
(SEQ ID NO: 17)
AADYGGGKYTRRQSEYEY;
(SEQ ID NO: 18)
AADYGGGRYTRRQSEYDY;
and
(SEQ ID NO: 19)
AADYGGGRYTRRQSEYEY.

30. The single domain antibody according to claim 25, wherein the amino acid sequence of CDR3 is selected from the group consisting of:
(SEQ ID NO: 5)
AADYGNKRYTRRQSEYEY;
(SEQ ID NO: 6)
AADYGTSRYTRRQSEYEY;
(SEQ ID NO: 11)
AADYGNKKYTRRQSEYEY;
(SEQ ID NO: 12)
AADYGNKRYTRRQSEYDY;
(SEQ ID NO: 13)
AADYGNKKYTRRQSEYDY;
(SEQ ID NO: 14)
AADYGTSKYTRRQSEYEY;
(SEQ ID NO: 15)
AADYGTSRYTRRQSEYDY;
and
(SEQ ID NO: 16)
AADYGTSKYTRRQSEYDY.

31. The single domain antibody according to claim 25, wherein the amino acid sequence of CDR3 is selected from the group consisting of:
(SEQ ID NO: 5)
AADYGNKRYTRRQSEYEY;
and
(SEQ ID NO: 6)
AADYGTSRYTRRQSEYEY.

32. The single domain antibody according to claim 25, wherein the single domain antibody is a VHH.

33. The single domain antibody according to claim 25, wherein the antibody comprises any one of SEQ ID NOs: 20-25 or a variant amino acid sequence having no more than 1, 2, 3, 4, 5, 6, 7, 8, 9, or 10 amino acid additions, deletions, substitutions, or combinations thereof within the sequence of SEQ ID NO: 20-25, said addition, deletion, or substitution being outside of CDR1, CDR2 and CDR3, with
SEQ ID NO: 20 being:
MA(E/D)VQLXXSGGGXVQXG(G/D)SLRLSC(S/A)(A/T)SGLT(F/
L)SRYAMGWFRQAPGNEREFVAVITASGRTTLYADS(V/L)KGRFTISRD
NAKNTVALQMQSLKPEDTAVYYCAADYGX₁X₂X₃YTRRQSEYX₄YWGQGT
QVTVSS(X)ₙAAA (SEQ ID NO: 20), with ₙ being 0-10;
X being any amino acid; X₁ and X₂ being any amino
acid, X₃ is K or R, and X₄ is D or E;
SEQ ID NO: 21 being:
MA(E/D)VQLVESGGGLVQAGDSLRLSCA(A/T)SGLTFSRYAMGWFRQA
PGNEREFVAVITASGRTTLYADSVKGRFTISRDNAKNTVALQMQSLKPED
TAVYYCAADYGX₁X₂RYTRRQSEYX₄YWGQGTQVTVSSAAA, with
X₁ and X₂ being any amino acid and X₄ being
D or E;
SEQ ID NO: 22 being:
MAEVQLVESGGGLVQAGDSLRLSCAASGLTLSRYAMGWFRQAPGNEREFV
AVITASGRTTLYADSLKGRFTISRDNAKNTVALQMQSLKPEDTAVYYCAA
DYGSGKYTRRQSEYDYWGQGTQVTVSSEPKTPKPQPAAA;
SEQ ID NO: 23 being:
MAEVQLVESGGGLVQAGDSLRLSCATSGLTFSRYAMGWFRQAPGNEREFV
AVITASGRTTLYADSVKGRFTISRDNAKNTVALQMQSLKPEDTAVYYCAA
DYGNKRYTRRQSEYEYWGQGTQVTVSSAAA;
SEQ ID NO: 24 being:
MADVQLVESGGGLVQAGDSLRLSCAASGLTFSRYAMGWFRQAPGNEREFV
AVITASGRTTLYADSVKGRFTISRDNAKNTVALQMQSLKPEDTAVYYCAA
DYGTSRYTRRQSEYEYWGQGTQVTVSSAAA;
and
SEQ ID NO: 25 being:
MAEVQLQASGGGSVQPGGSLRLSCSASGLTFSRYAMGWFRQAPGNEREFV
AVITASGRTTLYADSVKGRFTISRDNAKNTVALQMQSLKPEDTAVYYCAA
DYGGGRYTRRQSEYEYWGQGTQVTVSSAAA.

34. The single domain antibody according to claim 33, wherein the antibody comprises SEQ ID NO: 23 or 24 or a variant amino acid sequence having no more than 1, 2, 3, 4, 5, 6, 7, 8, 9, or 10 amino acid additions, deletions, substitutions, or combinations thereof within the sequence of SEQ ID NO: 23 or 24, said addition, deletion, or substitution being outside of CDR1, CDR2 and CDR3, with
SEQ ID NO: 23 being:
MAEVQLVESGGGLVQAGDSLRLSCATSGLTFSRYAMGWFRQAPGNEREFV
AVITASGRTTLYADSVKGRFTISRDNAKNTVALQMQSLKPEDTAVYYCAA
DYGNKRYTRRQSEYEYWGQGTQVTVSSAAA;
and
SEQ ID NO: 24 being:
MADVQLVESGGGLVQAGDSLRLSCAASGLTFSRYAMGWFRQAPGNEREFV
AVITASGRTTLYADSVKGRFTISRDNAKNTVALQMQSLKPEDTAVYYCAA
DYGTSRYTRRQSEYEYWGQGTQVTVSSAAA.

35. The single domain antibody according to claim 25 or a bivalent molecule comprising two of said single domain antibodies, wherein the single domain antibody or the bivalent molecule further comprises a detectable label, a detectable tag, an enzyme, a bioluminescent molecule, a fluorescent molecule, or a substitution by an amino acid suitable for being coupled to a detectable label.

36. The single domain antibody or the bivalent molecule according to claim 35, wherein the single domain antibody or the bivalent molecule is linked to the detectable label, a detectable tag, an enzyme, a bioluminescent protein or a fluorescent protein, as a fusion protein.

37. The single domain antibody or the bivalent molecule according to claim 36, wherein the fluorescent protein is selected from the group consisting of Green Fluorescent Protein, Enhanced Green Fluorescent Protein (EGFP), Enhanced Yellow Fluorescent Protein (EYFP), Venus, mVenus, Citrine, mCitrine, Cerulean, mCerulean, Orange Fluorescent Protein (OFP), mNeonGreen, moxNeonGreen, mCherry, mTagBFP, mTurquoise, mScarlet, mWasabi, mOrange, mStrawberry and dTomato.

38. A bivalent molecule comprising two single domain antibodies according to claim 25.

39. The bivalent molecule according to claim 38, wherein the two single domain antibodies are connected as a protein fusion or via a peptide linker.

40. The bivalent molecule according to claim 38, wherein the bivalent molecule comprises:
(SEQ ID NO: 26)
MA(E/D)VQLXXSGGGXVQXG(G/D)SLRLSC(S/A)(A/T)SGLT(F/L)SRYAMGWFR
QAPGNEREFVAVITASGRTTLYADS(V/L)KGRFTISRDNAKNTVALQMQSLKPEDTAVY
YCAADYGX₁X₂X₃YTRRQSEYX₄YWGQGTQVTVSS(X)ₙAA(A/-) - linker - MA(E/D)VQ
LXXSGGGXVQXG(G/D)SLRLSC(S/A)(A/T)SGLT(F/L)SRYAMGWFRQAPGNEREFVAVI
TASGRTTLYADS(V/L)KGRFTISRDNAKNTVALQMQSLKPEDTAVYYCAADYGX₁X₂X₃Y
TRRQSEYX₄YWGQGTQVTVSS(X)ₙAAA, wherein linker is a peptide
linker, ₙ is 0-10; X is any amino acid; X₁ and X₂ are any amino acid, X₃ is K or R, and X₄ is D or
E;
(SEQ ID NO: 27)
MA(E/D)VQLXXSGGGXVQXG(G/D)SLRLSC(S/A)(A/T)SGLT(F/L)SRYAMGWFR
QAPGNEREFVAVITASGRTTLYADS(V/L)KGRFTISRDNAKNTVALQMQSLKPEDTAVY
YCAADYGX₁X₂X₃YTRRQSEYX₄YWGQGTQVTVSS(X)ₙAA(A/-)GGGSGGGSGGGSMA
(E/D)VQLXXSGGGXVQXG(G/D)SLRLSC(S/A)(A/T)SGLT(F/L)SRYAMGWFRQAPGNER
EFVAVITASGRTTLYADS(V/L)KGRFTISRDNAKNTVALQMQSLKPEDTAVYYCAADYG
X₁X₂X₃YTRRQSEYX₄YWGQGTQVTVSS(X)ₙAAA, wherein ₙ is 0-10; X is
any amino acid; X₁ and X₂ are any amino acid, X₃ is K or R, and X₄ is D or E;
(SEQ ID NO: 28)
MA(E/D)VQLVESGGGLVQAGDSLRLSCA(A/T)SGLTFSRYAMGWFRQAPGNERE
FVAVITASGRTTLYADSVKGRFTISRDNAKNTVALQMQSLKPEDTAVYYCAADYGX₁X₂
RYTRRQSEYX₄YWGQGTQVTVSSAA(A/-) - linker - MA(E/D)VQLVESGGGLVQAGDSL
RLSCA(A/T)SGLTFSRYAMGWFRQAPGNEREFVAVITASGRTTLYADSVKGRFTISRDNA
KNTVALQMQSLKPEDTAVYYCAADYGX₁X₂RYTRRQSEYX₄YWGQGTQVTVSSAAA,
wherein linker is a peptide linker, and X₁ and X₂ are any amino acid and X₄
being D or E;
(SEQ ID NO: 29)
MA(E/D)VQLVESGGGLVQAGDSLRLSCA(A/T)SGLTFSRYAMGWFRQAPGNERE
FVAVITASGRTTLYADSVKGRFTISRDNAKNTVALQMQSLKPEDTAVYYCAADYGX1X2
RYTRRQSEYX4YWGQGTQVTVSSAA(A/-)GGGSGGGSGGGSMA(E/D)VQLVESGGGLVQ
AGDSLRLSCA(A/T)SGLTFSRYAMGWFRQAPGNEREFVAVITASGRTTLYADSVKGRFTI
SRDNAKNTVALQMQSLKPEDTAVYYCAADYGX₁X₂RYTRRQSEYX₄YWGQGTQVTVSS
AAA, with X₁ and X₂ being any amino acid and X₄ being D or E;
(SEQ ID NO: 30)
MAEVQLVESGGGLVQAGDSLRLSCAASGLTLSRYAMGWFRQAPGNEREFVAVI
TASGRTTLYADSLKGRFTISRDNAKNTVALQMQSLKPEDTAVYYCAADYGSGKYTRRQ
SEYDYWGQGTQVTVSSEPKTPKPQPAA (A/-) - linker - MAEVQLVESGGGLVQAGDSL
RLSCAASGLTLSRYAMGWFRQAPGNEREFVAVITASGRTTLYADSLKGRFTISRDNAKN
TVALQMQSLKPEDTAVYYCAADYGSGKYTRRQSEYDYWGQGTQVTVSSEPKTPKPQP
AAA, wherein linker is a peptide linker;
(SEQ ID NO: 31)
MAEVQLVESGGGLVQAGDSLRLSCAASGLTLSRYAMGWFRQAPGNEREFVAVI
TASGRTTLYADSLKGFTISRDNAKNTVALQMQSLKPEDTAVYYCAADYGSGKYTRRQS
EYDYWGQGTQVTVSSEPKTPKPQPAA (A/-) GGGSGGGSGGGSMAEVQLVESGGGLVQ
AGDSLRLSCAASGLTLSRYAMGWFRQAPGNEREFVAVITASGRTTLYADSLKGFTISRD
NAKNTVALQMQSLKPEDTAVYYCAADYGSGKYTRRQSEYDYWGQGTQVTVSSEPKTP
KPQPAAA;
(SEQ ID NO: 32)
MAEVQLVESGGGLVQAGDSLRLSCATSGLTFSRYAMGWFRQAPGNEREFVAVI
TASGRTTLYADSVKGRFTISRDNAKNTVALQMQSLKPEDTAVYYCAADYGNKRYTRR
QSEYEYWGQGTQVTVSSAA (A/-) - linker - MAEVQLVESGGGLVQAGDSLRLSCATSG
LTFSRYAMGWFRQAPGNEREFVAVITASGRTTLYADSVKGRFTISRDNAKNTVALQMQ
SLKPEDTAVYYCAADYGNKRYTRRQSEYEYWGQGTQVTVSSAAA,
wherein linker is a peptide linker;
(SEQ ID NO: 33)
MAEVQLVESGGGLVQAGDSLRLSCATSGLTFSRYAMGWFRQAPGNEREFVAVI
TASGRTTLYADSVKGRFTISRDNAKNTVALQMQSLKPEDTAVYYCAADYGNKRYTRR
QSEYEYWGQGTQVTVSSAA (A/-) GGGSGGGSGGGSMAEVQLVESGGGLVQAGDSLRL
SCATSGLTFSRYAMGWFRQAPGNEREFVAVITASGRTTLYADSVKGRFTISRDNAKNTV
ALQMQSLKPEDTAVYYCAADYGNKRYTRRQSEYEYWGQGTQVTVSSAAA;
(SEQ ID NO: 34)
MADVQLVESGGGLVQAGDSLRLSCAASGLTFSRYAMGWFRQAPGNEREFVAVI
TASGRTTLYADSVKGRFTISRDNAKNTVALQMQSLKPEDTAVYYCAADYGTSRYTRRQ
SEYEYWGQGTQVTVSSAA (A/-) - linker - MADVQLVESGGGLVQAGDSLRLSCAASG
LTFSRYAMGWFRQAPGNEREFVAVITASGRTTLYADSVKGRFTISRDNAKNTVALQMQ
SLKPEDTAVYYCAADYGTSRYTRRQSEYEYWGQGTQVTVSSAAA,
wherein linker is a peptide linker;
(SEQ ID NO: 35)
MADVQLVESGGGLVQAGDSLRLSCAASGLTFSRYAMGWFRQAPGNEREFVAVI
TASGRTTLYADSVKGRFTISRDNAKNTVALQMQSLKPEDTAVYYCAADYGTSRYTRRQ
SEYEYWGQGTQVTVSSAA (A/-) GGGSGGGSGGGSMADVQLVESGGGLVQAGDSLRLS
CAASGLTFSRYAMGWFRQAPGNEREFVAVITASGRTTLYADSVKGRFTISRDNAKNTV
ALQMQSLKPEDTAVYYCAADYGTSRYTRRQSEYEYWGQGTQVTVSSAAA;
(SEQ ID NO: 36)
MAEVQLQASGGGSVQPGGSLRLSCSASGLTFSRYAMGWFRQAPGNEREFVAVIT
ASGRTTLYADSVKGRFTISRDNAKNTVALQMQSLKPEDTAVYYCAADYGGGRYTRRQ
SEYEYWGQGTQVTVSSAA (A/-) - linker -MAEVQLQASGGGSVQPGGSLRLSCSASGLT
FSRYAMGWFRQAPGNEREFVAVITASGRTTLYADSVKGRFTISRDNAKNTVALQMQSL
KPEDTAVYYCAADYGGGRYTRRQSEYEYWGQGTQVTVSSAAA,
wherein linker is a peptide linker;
and
(SEQ ID NO: 37)
MAEVQLQASGGGSVQPGGSLRLSCSASGLTFSRYAMGWFRQAPGNEREFVAVIT
ASGRTTLYADSVKGRFTISRDNAKNTVALQMQSLKPEDTAVYYCAADYGGGRYTRRQ
SEYEYWGQGTQVTVSSAA (A/-) GGGSGGGSGGGSMAEVQLQASGGGSVQPGGSLRLS
CSASGLTFSRYAMGWFRQAPGNEREFVAVITASGRTTLYADSVKGRFTISRDNAKNTVA
LQMQSLKPEDTAVYYCAADYGGGRYTRRQSEYEYWGQGTQVTVSSAAA;
or a variant amino acid sequence having no more than 1, 2, 3, 4, 5, 6, 7, 8, 9, or 10 amino acid additions, deletions, substitutions, or combinations thereof, said addition, deletion, or substitution being outside of CDR1, CDR2 and CDR3.

41. The bivalent molecule according to claim 38, wherein the bivalent molecule comprises:
(SEQ ID NO: 32)
MAEVQLVESGGGLVQAGDSLRLSCATSGLTFSRYAMGWFRQAPGNEREFV
AVITASGRTTLYADSVKGRFTISRDNAKNTVALQMQSLKPEDTAVYYCAA
DYGNKRYTRRQSEYEYWGQGTQVTVSSAA (A/-) - linker - MAE
VQLVESGGGLVQAGDSLRLSCATSGLTFSRYAMGWFRQAPGNEREFVAVI
TASGRTTLYADSVKGRFTISRDNAKNTVALQMQSLKPEDTAVYYCAADYG
NKRYTRRQSEYEYWGQGTQVTVSSAAA,
wherein linker is a peptide linker;
(SEQ ID NO: 33)
MAEVQLVESGGGLVQAGDSLRLSCATSGLTFSRYAMGWFRQAPGNEREFV
AVITASGRTTLYADSVKGRFTISRDNAKNTVALQMQSLKPEDTAVYYCAA
DYGNKRYTRRQSEYEYWGQGTQVTVSSAA (A/-) GGGSGGGSGGGSMA
EVQLVESGGGLVQAGDSLRLSCATSGLTFSRYAMGWFRQAPGNEREFVAV
ITASGRTTLYADSVKGRFTISRDNAKNTVALQMQSLKPEDTAVYYCAADY
GNKRYTRRQSEYEYWGQGTQVTVSSAAA;
(SEQ ID NO: 34)
MADVQLVESGGGLVQAGDSLRLSCAASGLTFSRYAMGWFRQAPGNEREFV
AVITASGRTTLYADSVKGRFTISRDNAKNTVALQMQSLKPEDTAVYYCAA
DYGTSRYTRRQSEYEYWGQGTQVTVSSAA (A/-) - linker - MAD
VQLVESGGGLVQAGDSLRLSCAASGLTFSRYAMGWFRQAPGNEREFVAVI
TASGRTTLYADSVKGRFTISRDNAKNTVALQMQSLKPEDTAVYYCAADYG
TSRYTRRQSEYEYWGQGTQVTVSSAAA,
wherein linker is a peptide linker;
(SEQ ID NO: 35)
MADVQLVESGGGLVQAGDSLRLSCAASGLTFSRYAMGWFRQAPGNEREFV
AVITASGRTTLYADSVKGRFTISRDNAKNTVALQMQSLKPEDTAVYYCAA
DYGTSRYTRRQSEYEYWGQGTQVTVSSAA (A/-) GGGSGGGSGGGSMA
DVQLVESGGGLVQAGDSLRLSCAASGLTFSRYAMGWFRQAPGNEREFVAV
ITASGRTTLYADSVKGRFTISRDNAKNTVALQMQSLKPEDTAVYYCAADY
GTSRYTRRQSEYEYWGQGTQVTVSSAAA;
or a variant amino acid sequence having no more than 1, 2, 3, 4, 5, 6, 7, 8, 9, or 10 amino acid additions, deletions, substitutions, or combinations thereof, said addition, deletion, or substitution being outside of CDR1, CDR2 and CDR3.

42. A nucleic acid, an expression cassette or an expression vector encoding a single domain antibody according to claim 25 or a bivalent molecule comprising two of said single domain antibodies.

43. A host cell comprising a nucleic acid, an expression cassette or an expression vector according to claim 42.

44. A method for detecting H2AX phosphorylated on S139 (γ-H2AX) in a cell comprising contacting the cell with a single domain antibody according to claim 25 or a bivalent molecule comprising two of said single domain antibodies and detecting and/quantifying the presence of the single domain antibody or bivalent molecule, thereby detecting γ-H2AX or γ-H2AX foci.


SEQUENCE LISTING (84 sequences)

SEQ ID NO: 1 - protein, 8 aa, Artificial
  CDR1
  MISC_FEATURE (4)..(4): Xaa is L or F
GLTXSRYA

SEQ ID NO: 2 - protein, 8 aa, artificial
  CDR2
ITASGRTT

SEQ ID NO: 3 - protein, 18 aa, artificial
  CDR3
  MISC_FEATURE (6)..(7): Xaa is any amino acid
  MISC_FEATURE (8)..(8): Xaa is K or R
  MISC_FEATURE (17)..(17): Xaa is D or E
AADYGXXXYTRRQSEYXY

SEQ ID NO: 4 - protein, 18 aa, artificial
  CDR3
AADYGSGKYTRRQSEYDY

SEQ ID NO: 5 - protein, 18 aa, artificial
  CDR3
AADYGNKRYTRRQSEYEY

SEQ ID NO: 6 - protein, 18 aa, artificial
  CDR3
AADYGTSRYTRRQSEYEY

SEQ ID NO: 7 - protein, 18 aa, artificial
  CDR3
AADYGGGRYTRRQSEYEY

SEQ ID NO: 8 - protein, 18 aa, artificial
  CDR3
AADYGSGRYTRRQSEYDY

SEQ ID NO: 9 - protein, 18 aa, artificial
  CDR3
AADYGSGKYTRRQSEYEY

SEQ ID NO: 10 - protein, 18 aa, artificial
  CDR3
AADYGSGRYTRRQSEYEY

SEQ ID NO: 11 - protein, 18 aa, artificial
  CDR3
AADYGNKKYTRRQSEYEY

SEQ ID NO: 12 - protein, 18 aa, artificial
  CDR3
AADYGNKRYTRRQSEYDY

SEQ ID NO: 13 - protein, 18 aa, artificial
  CDR3
AADYGNKKYTRRQSEYDY

SEQ ID NO: 14 - protein, 18 aa, artificial
  CDR3
AADYGTSKYTRRQSEYEY

SEQ ID NO: 15 - protein, 18 aa, artificial
  CDR3
AADYGTSRYTRRQSEYDY

SEQ ID NO: 16 - protein, 18 aa, artificial
  CDR3
AADYGTSKYTRRQSEYDY

SEQ ID NO: 17 - protein, 18 aa, artificial
  CDR3
AADYGGGKYTRRQSEYEY

SEQ ID NO: 18 - protein, 18 aa, artificial
  CDR3
AADYGGGRYTRRQSEYDY

SEQ ID NO: 19 - protein, 18 aa, artificial
  CDR3
AADYGGGRYTRRQSEYEY

SEQ ID NO: 20 - protein, 131 aa, artificial
  single domain antibody
  MISC_FEATURE (3)..(3): Xaa is Glu or Asp
  MISC_FEATURE (7)..(8): Xaa is any amino acid
  MISC_FEATURE (13)..(13): Xaa is any amino acid
  MISC_FEATURE (16)..(16): Xaa is any amino acid
  MISC_FEATURE (18)..(18): Xaa is Gly or Asp
  MISC_FEATURE (25)..(25): Xaa is Ser or Ala
  MISC_FEATURE (26)..(26): Xaa is Ala or Thr
  MISC_FEATURE (31)..(31): Xaa is Phe or Leu
  MISC_FEATURE (66)..(66): Xaa is Val or Leu
  MISC_FEATURE (104)..(105): Xaa is any amino acid
  MISC_FEATURE (106)..(106): Xaa is Lys or Arg
  MISC_FEATURE (115)..(115): Xaa is Asp or Glu
  MISC_FEATURE (128)..(128): Xaa is 0-10 amino acids, the amino acids being any amino acid
MAXVQLXXSGGGXVQXGXSLRLSCXXSGLTXSRYAMGWFRQAPGNEREFVAVITASGRTTLYADSXKGRFTISRDNAKNTVALQMQSLKPEDTAVYYCAADYGXXXYTRRQSEYXYWGQGTQVTVSSXAAA

SEQ ID NO: 21 - protein, 130 aa, artificial
  single domain antibody
  MISC_FEATURE (3)..(3): Xaa is Glu or Asp
  MISC_FEATURE (26)..(26): Xaa is Ala or Thr
  MISC_FEATURE (104)..(105): Xaa is any amino acid
  MISC_FEATURE (115)..(115): Xaa is Asp or Glu
MAXVQLVESGGGLVQAGDSLRLSCAXSGLTFSRYAMGWFRQAPGNEREFVAVITASGRTTLYADSVKGRFTISRDNAKNTVALQMQSLKPEDTAVYYCAADYGXXRYTRRQSEYXYWGQGTQVTVSSAAA

SEQ ID NO: 22 - protein, 139 aa, artificial
  single domain antibody
MAEVQLVESGGGLVQAGDSLRLSCAASGLTLSRYAMGWFRQAPGNEREFVAVITASGRTTLYADSLKGRFTISRDNAKNTVALQMQSLKPEDTAVYYCAADYGSGKYTRRQSEYDYWGQGTQVTVSSEPKTPKPQPAAA

SEQ ID NO: 23 - protein, 130 aa, artificial
  single domain antibody
MAEVQLVESGGGLVQAGDSLRLSCATSGLTFSRYAMGWFRQAPGNEREFVAVITASGRTTLYADSVKGRFTISRDNAKNTVALQMQSLKPEDTAVYYCAADYGNKRYTRRQSEYEYWGQGTQVTVSSAAA

SEQ ID NO: 24 - protein, 130 aa, artificial
  single domain antibody
MADVQLVESGGGLVQAGDSLRLSCAASGLTFSRYAMGWFRQAPGNEREFVAVITASGRTTLYADSVKGRFTISRDNAKNTVALQMQSLKPEDTAVYYCAADYGTSRYTRRQSEYEYWGQGTQVTVSSAAA

SEQ ID NO: 25 - protein, 130 aa, artificial
  single domain antibody
MAEVQLQASGGGSVQPGGSLRLSCSASGLTFSRYAMGWFRQAPGNEREFVAVITASGRTTLYADSVKGRFTISRDNAKNTVALQMQSLKPEDTAVYYCAADYGGGRYTRRQSEYEYWGQGTQVTVSSAAA

SEQ ID NO: 26 - protein, 263 aa, artificial
  bivalent protein
  MISC_FEATURE (3)..(3): Xaa is Glu or Asp
  MISC_FEATURE (7)..(8): Xaa is any amino acid
  MISC_FEATURE (13)..(13): Xaa is any amino acid
  MISC_FEATURE (16)..(16): Xaa is any amino acid
  MISC_FEATURE (18)..(18): Xaa is Gly or Asp
  MISC_FEATURE (25)..(25): Xaa is Ser or Ala
  MISC_FEATURE (26)..(26): Xaa is Ala or Thr
  MISC_FEATURE (31)..(31): Xaa is Phe or Leu
  MISC_FEATURE (66)..(66): Xaa is Val or Leu
  MISC_FEATURE (104)..(105): Xaa is any amino acid
  MISC_FEATURE (106)..(106): Xaa is Lys or Arg
  MISC_FEATURE (115)..(115): Xaa is Asp or Glu
  MISC_FEATURE (128)..(128): Xaa is 0-10 amino acids, the amino acids being any amino acid
  MISC_FEATURE (131)..(131): Xaa is Ala or absent
  MISC_FEATURE (132)..(132): Xaa is a linker
  MISC_FEATURE (135)..(135): Xaa is Glu or Asp
  MISC_FEATURE (139)..(140): Xaa is any amino acid
  MISC_FEATURE (145)..(145): Xaa is any amino acid
  MISC_FEATURE (148)..(148): Xaa is any amino acid
  MISC_FEATURE (150)..(150): Xaa is Gly or Asp
  MISC_FEATURE (157)..(157): Xaa is Ser or Ala
  MISC_FEATURE (158)..(158): Xaa is Thr or Ala
  MISC_FEATURE (163)..(163): Xaa is Phe or Leu
  MISC_FEATURE (198)..(198): Xaa is Val or Leu
  MISC_FEATURE (236)..(237): Xaa is any amino acid
  MISC_FEATURE (238)..(238): Xaa is Lys or Arg
  MISC_FEATURE (247)..(247): Xaa is Glu or Asp
  MISC_FEATURE (260)..(260): Xaa is 0-10 amino acids, the amino acid being any amino acid
MAXVQLXXSGGGXVQXGXSLRLSCXXSGLTXSRYAMGWFRQAPGNEREFVAVITASGRTTLYADSXKGRFTISRDNAKNTVALQMQSLKPEDTAVYYCAADYGXXXYTRRQSEYXYWGQGTQVTVSSXAAXXMAXVQLXXSGGGXVQXGXSLRLSCXXSGLTXSRYAMGWFRQAPGNEREFVAVITASGRTTLYADSXKGRFTISRDNAKNTVALQMQSLKPEDTAVYYCAADYGXXXYTRRQSEYXYWGQGTQVTVSSXAAA

SEQ ID NO: 27 - protein, 274 aa, artificial
  bivalent protein
  MISC_FEATURE (3)..(3): Xaa is Glu or Asp
  MISC_FEATURE (7)..(8): Xaa is any amino acid
  MISC_FEATURE (13)..(13): Xaa is any amino acid
  MISC_FEATURE (16)..(16): Xaa is any amino acid
  MISC_FEATURE (18)..(18): Xaa is Gly or Asp
  MISC_FEATURE (25)..(25): Xaa is Ser or Ala
  MISC_FEATURE (26)..(26): Xaa is Thr or Ala
  MISC_FEATURE (31)..(31): Xaa is Phe or Leu
  MISC_FEATURE (66)..(66): Xaa is Val or Leu
  MISC_FEATURE (104)..(105): Xaa is any amino acid
  MISC_FEATURE (106)..(106): Xaa is Lys or Arg
  MISC_FEATURE (115)..(115): Xaa is Glu or Asp
  MISC_FEATURE (128)..(128): Xaa is 0-10 amino acids, the amino acids being any amino acid
  MISC_FEATURE (131)..(131): Xaa is Ala or absent
  MISC_FEATURE (146)..(146): Xaa is Glu or Asp
  MISC_FEATURE (150)..(151): Xaa is any amino acid
  MISC_FEATURE (156)..(156): Xaa is any amino acid
  MISC_FEATURE (159)..(159): Xaa is any amino acid
  MISC_FEATURE (161)..(161): Xaa is Gly or Asp
  MISC_FEATURE (168)..(168): Xaa is Ser or Ala
  MISC_FEATURE (169)..(169): Xaa is Thr or Ala
  MISC_FEATURE (174)..(174): Xaa is Phe or Leu
  MISC_FEATURE (209)..(209): Xaa is Val or Leu
  MISC_FEATURE (247)..(248): Xaa is any amino acid
  MISC_FEATURE (249)..(249): Xaa is Lys or Arg
  MISC_FEATURE (258)..(258): Xaa is Glu or Asp
  MISC_FEATURE (271)..(271): Xaa is 0-10 amino acids, the amino acids being any amino acid
MAXVQLXXSGGGXVQXGXSLRLSCXXSGLTXSRYAMGWFRQAPGNEREFVAVITASGRTTLYADSXKGRFTISRDNAKNTVALQMQSLKPEDTAVYYCAADYGXXXYTRRQSEYXYWGQGTQVTVSSXAAXGGGSGGGSGGGSMAXVQLXXSGGGXVQXGXSLRLSCXXSGLTXSRYAMGWFRQAPGNEREFVAVITASGRTTLYADSXKGRFTISRDNAKNTVALQMQSLKPEDTAVYYCAADYGXXXYTRRQSEYXYWGQGTQVTVSSXAAA

SEQ ID NO: 28 - protein, 261 aa, artificial
  bivalent protein
  MISC_FEATURE (3)..(3): Xaa is Glu or Asp
  MISC_FEATURE (26)..(26): Xaa is Ala or Thr
  MISC_FEATURE (104)..(105): Xaa is any amino acid
  MISC_FEATURE (115)..(115): Xaa is Glu or Asp
  MISC_FEATURE (130)..(130): Xaa is Ala or absent
  MISC_FEATURE (131)..(131): Xaa is a linker
  MISC_FEATURE (134)..(134): Xaa is Glu or Asp
  MISC_FEATURE (157)..(157): Xaa is Ala or Thr
  MISC_FEATURE (235)..(236): Xaa is any amino acid
  MISC_FEATURE (246)..(246): Xaa is Glu or Asp
MAXVQLVESGGGLVQAGDSLRLSCAXSGLTFSRYAMGWFRQAPGNEREFVAVITASGRTTLYADSVKGRFTISRDNAKNTVALQMQSLKPEDTAVYYCAADYGXXRYTRRQSEYXYWGQGTQVTVSSAAXXMAXVQLVESGGGLVQAGDSLRLSCAXSGLTFSRYAMGWFRQAPGNEREFVAVITASGRTTLYADSVKGRFTISRDNAKNTVALQMQSLKPEDTAVYYCAADYGXXRYTRRQSEYXYWGQGTQVTVSSAAA

SEQ ID NO: 29 - protein, 272 aa, artificial
  bivalent protein
  MISC_FEATURE (3)..(3): Xaa is Glu or Asp
  MISC_FEATURE (26)..(26): Xaa is Ala or Thr
  MISC_FEATURE (104)..(105): Xaa is any amino acid
  MISC_FEATURE (115)..(115): Xaa is Glu or Asp
  MISC_FEATURE (130)..(130): Xaa is Ala or absent
  MISC_FEATURE (145)..(145): Xaa is Glu or Asp
  MISC_FEATURE (168)..(168): Xaa is Ala or Thr
  MISC_FEATURE (246)..(247): Xaa is any amino acid
  MISC_FEATURE (257)..(257): Xaa is Glu or Asp
MAXVQLVESGGGLVQAGDSLRLSCAXSGLTFSRYAMGWFRQAPGNEREFVAVITASGRTTLYADSVKGRFTISRDNAKNTVALQMQSLKPEDTAVYYCAADYGXXRYTRRQSEYXYWGQGTQVTVSSAAXGGGSGGGSGGGSMAXVQLVESGGGLVQAGDSLRLSCAXSGLTFSRYAMGWFRQAPGNEREFVAVITASGRTTLYADSVKGRFTISRDNAKNTVALQMQSLKPEDTAVYYCAADYGXXRYTRRQSEYXYWGQGTQVTVSSAAA

SEQ ID NO: 30 - protein, 279 aa, artificial
  bivalent protein
  MISC_FEATURE (139)..(139): Xaa is Ala or absent
  MISC_FEATURE (140)..(140): Xaa is a linker
MAEVQLVESGGGLVQAGDSLRLSCAASGLTLSRYAMGWFRQAPGNEREFVAVITASGRTTLYADSLKGRFTISRDNAKNTVALQMQSLKPEDTAVYYCAADYGSGKYTRRQSEYDYWGQGTQVTVSSEPKTPKPQPAAXXMAEVQLVESGGGLVQAGDSLRLSCAASGLTLSRYAMGWFRQAPGNEREFVAVITASGRTTLYADSLKGRFTISRDNAKNTVALQMQSLKPEDTAVYYCAADYGSGKYTRRQSEYDYWGQGTQVTVSSEPKTPKPQPAAA

SEQ ID NO: 31 - protein, 290 aa, artificial
  bivalent protein
  MISC_FEATURE (139)..(139): Xaa is Ala or absent
MAEVQLVESGGGLVQAGDSLRLSCAASGLTLSRYAMGWFRQAPGNEREFVAVITASGRTTLYADSLKGRFTISRDNAKNTVALQMQSLKPEDTAVYYCAADYGSGKYTRRQSEYDYWGQGTQVTVSSEPKTPKPQPAAXGGGSGGGSGGGSMAEVQLVESGGGLVQAGDSLRLSCAASGLTLSRYAMGWFRQAPGNEREFVAVITASGRTTLYADSLKGRFTISRDNAKNTVALQMQSLKPEDTAVYYCAADYGSGKYTRRQSEYDYWGQGTQVTVSSEPKTPKPQPAAA

SEQ ID NO: 32 - protein, 261 aa, artificial
  bivalent protein
  MISC_FEATURE (130)..(130): Xaa is Ala or absent
  MISC_FEATURE (131)..(131): Xaa is a linker
MAEVQLVESGGGLVQAGDSLRLSCATSGLTFSRYAMGWFRQAPGNEREFVAVITASGRTTLYADSVKGRFTISRDNAKNTVALQMQSLKPEDTAVYYCAADYGNKRYTRRQSEYEYWGQGTQVTVSSAAXXMAEVQLVESGGGLVQAGDSLRLSCATSGLTFSRYAMGWFRQAPGNEREFVAVITASGRTTLYADSVKGRFTISRDNAKNTVALQMQSLKPEDTAVYYCAADYGNKRYTRRQSEYEYWGQGTQVTVSSAAA

SEQ ID NO: 33 - protein, 272 aa, artificial
  bivalent protein
  MISC_FEATURE (130)..(130): Xaa is Ala or absent
MAEVQLVESGGGLVQAGDSLRLSCATSGLTFSRYAMGWFRQAPGNEREFVAVITASGRTTLYADSVKGRFTISRDNAKNTVALQMQSLKPEDTAVYYCAADYGNKRYTRRQSEYEYWGQGTQVTVSSAAXGGGSGGGSGGGSMAEVQLVESGGGLVQAGDSLRLSCATSGLTFSRYAMGWFRQAPGNEREFVAVITASGRTTLYADSVKGRFTISRDNAKNTVALQMQSLKPEDTAVYYCAADYGNKRYTRRQSEYEYWGQGTQVTVSSAAA

SEQ ID NO: 34 - protein, 261 aa, artificial
  bivalent protein
  MISC_FEATURE (130)..(130): Xaa is Ala or absent
  MISC_FEATURE (131)..(131): Xaa is a linker
MADVQLVESGGGLVQAGDSLRLSCAASGLTFSRYAMGWFRQAPGNEREFVAVITASGRTTLYADSVKGRFTISRDNAKNTVALQMQSLKPEDTAVYYCAADYGTSRYTRRQSEYEYWGQGTQVTVSSAAXXMADVQLVESGGGLVQAGDSLRLSCAASGLTFSRYAMGWFRQAPGNEREFVAVITASGRTTLYADSVKGRFTISRDNAKNTVALQMQSLKPEDTAVYYCAADYGTSRYTRRQSEYEYWGQGTQVTVSSAAA

SEQ ID NO: 35 - protein, 272 aa, artificial
  bivalent protein
  MISC_FEATURE (130)..(130): Xaa is Ala or absent
MADVQLVESGGGLVQAGDSLRLSCAASGLTFSRYAMGWFRQAPGNEREFVAVITASGRTTLYADSVKGRFTISRDNAKNTVALQMQSLKPEDTAVYYCAADYGTSRYTRRQSEYEYWGQGTQVTVSSAAXGGGSGGGSGGGSMADVQLVESGGGLVQAGDSLRLSCAASGLTFSRYAMGWFRQAPGNEREFVAVITASGRTTLYADSVKGRFTISRDNAKNTVALQMQSLKPEDTAVYYCAADYGTSRYTRRQSEYEYWGQGTQVTVSSAAA

SEQ ID NO: 36 - protein, 261 aa, artificial
  bivalent protein
  MISC_FEATURE (130)..(130): Xaa is Ala or absent
  MISC_FEATURE (131)..(131): Xaa is a linker
MAEVQLQASGGGSVQPGGSLRLSCSASGLTFSRYAMGWFRQAPGNEREFVAVITASGRTTLYADSVKGRFTISRDNAKNTVALQMQSLKPEDTAVYYCAADYGGGRYTRRQSEYEYWGQGTQVTVSSAAXXMAEVQLQASGGGSVQPGGSLRLSCSASGLTFSRYAMGWFRQAPGNEREFVAVITASGRTTLYADSVKGRFTISRDNAKNTVALQMQSLKPEDTAVYYCAADYGGGRYTRRQSEYEYWGQGTQVTVSSAAA

SEQ ID NO: 37 - protein, 272 aa, artificial
  bivalent protein
  MISC_FEATURE (130)..(130): Xaa is Ala or absent
MAEVQLQASGGGSVQPGGSLRLSCSASGLTFSRYAMGWFRQAPGNEREFVAVITASGRTTLYADSVKGRFTISRDNAKNTVALQMQSLKPEDTAVYYCAADYGGGRYTRRQSEYEYWGQGTQVTVSSAAXGGGSGGGSGGGSMAEVQLQASGGGSVQPGGSLRLSCSASGLTFSRYAMGWFRQAPGNEREFVAVITASGRTTLYADSVKGRFTISRDNAKNTVALQMQSLKPEDTAVYYCAADYGGGRYTRRQSEYEYWGQGTQVTVSSAAA

SEQ ID NO: 38 - protein, 226 aa, artificial
  dTomato
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFL

SEQ ID NO: 39 - DNA, 29 bp, artificial
  VHBACK-A6
gatgtgcagctgcaggcgtctggrggagg

SEQ ID NO: 40 - DNA, 21 bp, artificial
  CH2FORTA4
cgccatcaaggtaccagttga

SEQ ID NO: 41 - DNA, 22 bp, artificial
  CALL001
gtcctggctgctctctacaagg

SEQ ID NO: 42 - DNA, 23 bp, artificial
  CALL002
ggtacgtgctgttgaactgttcc

SEQ ID NO: 43 - DNA, 23 bp, artificial
  AlpVh-L
ctgagcttggtggtcctggctgc

SEQ ID NO: 44 - DNA, 23 bp, artificial
  Bq-lead-lg-F
gtcctggctgctctwytacargg

SEQ ID NO: 45 - DNA, 23 bp, artificial
  Bq-CH2-ca2-R
ggtacgtgctgttgaactgttcc

SEQ ID NO: 46 - DNA, 39 bp, artificial
  SM017
ccagccggccatggctcaggtgcagctggtggagtctgg

SEQ ID NO: 47 - DNA, 39 bp, artificial; identical to SEQ ID NO: 46
  SM018

SEQ ID NO: 48 - DNA, 44 bp, artificial
  VHBACKA4
catgccatgactcggggcccagccggccatggcgakgtscagct

SEQ ID NO: 49 - DNA, 44 bp, artificial; identical to SEQ ID NO: 48
  VHFOR36

SEQ ID NO: 50 - DNA, 48 bp, artificial
  Bq-FR1-long-F
  misc_feature (46)..(46): n is a, c, g, or t
gtcattggcccagccggccatggctcagktgcagctcgtggagtcngg

SEQ ID NO: 51 - DNA, 35 bp, artificial
  Bq-Hin-short-F
gacattgcggccgcgctggggtcttcgctgtggtg

SEQ ID NO: 52 - DNA, 38 bp, artificial
  Bq-Hin-long-R
gacattgcggccgctggttgtggttttggtgtcttggg

SEQ ID NO: 53 - DNA, 33 bp, artificial
  E6T-For
ctagtatgtttcaggatccagaacgtccgcgcg

SEQ ID NO: 54 - DNA, 36 bp, artificial
  E6T-Back
ctagcgcgcggacgttcctgcggatcctgaaacata

SEQ ID NO: 55 - DNA, 38 bp, artificial
  VHH-BspH1-For
aacgaactcatgactcagktgcagctcgtggagtcggg

SEQ ID NO: 56 - DNA, 38 bp, artificial
  VHH-BspH1-Deg
aacgaactcatgacysabbtscagctsswgsmgtcvcc

SEQ ID NO: 57 - DNA, 35 bp, artificial
  VHH-Not1-short
ggactagttgcggccgctgaggagacggtgacctg

SEQ ID NO: 58 - DNA, 41 bp, artificial
  VHH-Not1-long
ggactagttgcggccgctggttgtggttttggtgtttcggg

SEQ ID NO: 59 - DNA, 20 bp, artificial
  pETOM-For
ggagaccacaacggtttccc

SEQ ID NO: 60 - DNA, 20 bp, artificial
  pET-Rev
ttcgggctttgttagcagcc

SEQ ID NO: 61 - DNA, 20 bp, artificial
  b-actin-For
ggctcacagcgcgcccggct

SEQ ID NO: 62 - DNA, 65 bp, artificial
  G4S-Rev
cgatccgccaccgccgctgccacctccgcctgaaccgcctccaccggccgctgaggagacggtga

SEQ ID NO: 63 - DNA, 65 bp, artificial
  G4S-For
ggtggaggcggttcaggcggaggtggcagcggcggtggcggatcgatgactgaggtgcagctggt

SEQ ID NO: 64 - DNA, 20 bp, artificial
  E6Tag-Rev
tcctgcggatcctgaaacat

SEQ ID NO: 65 - DNA, 29 bp, artificial
  C6-Cys-Rev
aaaaaatgcggccgcacatgaggagacgg

SEQ ID NO: 66 - DNA, 26 bp, artificial
  C6-Mut-For
gcagatataccgcgatacagtctgag

SEQ ID NO: 67 - DNA, 26 bp, artificial
  C6-Mut-Rev
ctcagactgtatcgcggtatatctgc

SEQ ID NO: 68 - DNA, 21 bp, artificial
  mCher-Rev
cttgtacagctcgtccatgcc

SEQ ID NO: 69 - DNA, 33 bp, artificial
  mCher-pET-For
atttatgctagcggagggatggtgagcaagggc

SEQ ID NO: 70 - DNA, 34 bp, artificial
  mCher-pET-Rev
atttatgctagcactacccttgtacagctcgtcc

SEQ ID NO: 71 - DNA, 34 bp, artificial
  dTo-Bam-For
gcgcatggatcctatggtgagcaagggcgaggag

SEQ ID NO: 72 - DNA, 32 bp, artificial
  dTo-Bam-Rev
gcgcgcggatccccggtgctgccggtgccatg

SEQ ID NO: 73 - DNA, 42 bp, artificial
  E6T-Hind-For
ctagtatgtttcaggatccgcaggaacgtccgcgcaagcttg

SEQ ID NO: 74 - DNA, 42 bp, artificial
  E6T-Hind-Rev
ctagcaagcttgcgcggacgttcctgcggatcctgaaacata

SEQ ID NO: 75 - DNA, 33 bp, artificial
  mCherry-For
atttataagcttagtgggatggtgagcaagggc

SEQ ID NO: 76 - DNA, 34 bp, artificial
  mCherry-Rev
atttatgaattctcattacttgtacagctcgtcc

SEQ ID NO: 77 - DNA, 51 bp, artificial
  NESPKIa-Hind-For
agcttaacgagctcgctctcaaactcgctggactcgacatcaacaagacca

SEQ ID NO: 78 - DNA, 51 bp, artificial
  NESPKIa-Hind-Rev
agcttggtcttgttgatgtcgagtccagcgagtttgagagcgagctcgtta

SEQ ID NO: 79 - protein, 138 aa, artificial
  A4
MAEVQLVESGGGLVQAGDSLRLSCAASGLTLSRYAMGWFRQAPGNEREFVAVITASGRTTLYADSLKGFTISRDNAKNTVALQMQSLKPEDTAVYYCAADYGSGKYTRRQSEYDYWGQGTQVTVSSEPKTPKPQPAAA

SEQ ID NO: 80 - protein, 129 aa, artificial
  A9
MAEVQLVESGGGLVQAGDSLRLSCATSGLTFSRYAMGWFRQAPGNEREFVAVITASGRTTLYADSVKRFTISRDNAKNTVALQMQSLKPEDTAVYYCAADYGNKRYTRRQSEYEYWGQGTQVTVSSAAA

SEQ ID NO: 81 - protein, 129 aa, artificial
  C6
MADVQLVESGGGLVQAGDSLRLSCAASGLTFSRYAMGWFRQAPGNEREFVAVITASGRTTLYADSVKRFTISRDNAKNTVALQMQSLKPEDTAVYYCAADYGTSRYTRRQSEYEYWGQGTQVTVSSAAA

SEQ ID NO: 82 - protein, 129 aa, artificial
  G2
MAEVQLQASGGGSVQPGGSLRLSCSASGLTFSRYAMGWFRQAPGNEREFVAVITASGRTTLYADSVKRFTISRDNAKNTVALQMQSLKPEDTAVYYCAADYGGGRYTRRQSEYEYWGQGTQVTVSSAAA

SEQ ID NO: 83 - protein, 143 aa, Homo sapiens
MSGRGKTGGKARAKAKSRSSRAGLQFPVGRVHRLLRKGHYAERVGAGAPVYLAAVLEYLTAEILELAGNAARDNKKTRIIPRHLQLAIRNDEELNKLLGGVTIAQGGVLPNIQAVLLPKKTSATVGPKAPSGGKKATQASQEY

SEQ ID NO: 84 - protein, 143 aa, Mus musculus
MSGRGKTGGKARAKAKSRSSRAGLQFPVGRVHRLLRKGHYAERVGAGAPVYLAAVLEYLTAEILELAGNAARDNKKTRIIPRHLQLAIRNDEELNKLLGGVTIAQGGVLPNIQAVLLPKKSSATVGPKAPAVGKKASQASQEY